(19) United States
(12) Patent Application Publication
(10) Pub. No.: US 2008/0139457 A1
(43) Pub. Date: Jun. 12, 2008

(54) Therapeutic compositions comprising chorionic gonadotropins and HMG CoA reductase inhibitors

(75) Inventor: Paul Dent, Glenallen, VA (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: Virginia Commonwealth University, Richmond, VA (US)

(21) Appl. No.: 11/521,213

(22) Filed: Sep. 14, 2006

Related U.S. Application Data
(60) Provisional application No. 60/717,205, filed on Sep. 16, 2005.

(51) Int. Cl. A61K 38/24; A61P 35/00

(52) U.S. Cl. 514/8

(57) ABSTRACT

The invention provides improved methods and compositions for the treatment of cancer in a subject comprising administering to said subject a therapeutically effective amount of a composition comprising a chorionic gonadotropin or a therapeutically active fragment or analogue thereof in combination with an HMG CoA reductase inhibitor. The compositions and methods may also comprise geranylgeranyltransferase inhibitors (GGTI) and farnesyltransferase inhibitors (FTI) instead of or in addition to the HMG CoA reductase inhibitors of the invention.

### CROSS REFERENCE TO RELATED APPLICATIONS

This application claims benefit of U.S. Provisional Application Ser. No. 60/717,205 filed Sep. 16, 2005.

### STATEMENT OF GOVERNMENTAL INTERESTS

This invention was made with government support under Department of Defense Award (DAMD17-03-1-0262) and PHS(R01-CA88906, R01-DK52825, P01-CA72955, P01-CA104177, R01-CA108520, P01-CA72955, R01-CA63753 and R01-CA77141). The government has certain rights in this invention.

### BACKGROUND OF THE DISCLOSURE

Human chorionic gonadotropin belongs to a family of glycoprotein hormones, human luteinizing hormone (lutropin, hLH), follitropin (FSH), and thyrotropin (TSH). Each of these hormones is composed of two dissimilar, noncovalently bound subunits, α- and β-. The hormones share a common α-subunit, while the β-subunits differ slightly in length and amino acid sequence (Ryan et al. (1988) FASEB J. 2: 2661-2669; Ward et al. in Reproduction in Domestic Animals, 4th ed., Cuppos, P T, ed., pp. 25-80, Academic Press, NY (1991). The most closely related of the β-subunits are those of hCG and hLH, which are 85% identical, except for an approximately 20 amino acid extension on the carboxy terminus of hCG. Indeed, hCG and hLH act through a common receptor (Loosfelt et al. (1989) Science 245: 525-528; McFarland et al. (1989) Science 245: 494-499) and elicit identical biological responses (Pierce and Parsons (1981) Ann. Rev. Biochem. 50: 466; Strickland et al. in Luteinizing hormone action and receptors, M. Ascoli, Ed., CRC Press, Boca Raton Fla., 1985, p. 1).

Human chorionic gonadotropin (hCG) binds to the luteinizing hormone (LH) receptor, and is involved in the normal growth and differentiation of multiple male and female cell types in organs involved with sexual reproduction (Hong et al, Mol Endocrinol. 13: 1285-1294 (1999) and Filicori et al. Fertil Steril. 84: 275-284 (2005)). Clinically, hCG is often used as a therapeutic in children with low levels of hCG during puberty and exhibiting a lack of appropriate physical development and administration of this hormone acts to promote sexual maturation ((Styne D M, Endocrinol Metab Clin North Am. 20: 43-69 (1991) (Soliman et al., Metabolism 54: 15-23 (2005) and Delemarre-van de Waal, Eur J Endocrinol; 151: U89-94 (2004)).

However, prolonged over-expression of hCG in adults, particularly in females, has been linked to the development of tumors e.g. gestational trophoblastic diseases; choriocarcinoma/germ-cell tumors; osteosarcoma; bladder cancer, prostate cancer (Sheaff et al., J Clin Pathol. 49: 329-332 (1996) and Huhtaniemi et al., Mol Cell Endocrinol. 234: 117-126 (2005)).

In contrast, at least one study has argued that elevated hCG levels may be cancer-preventative for breast cancer (Cole et al., Gynecol Oncol. 102: 145-150 (2006) and Salhab et al., Int J Fertil Womens Med. 50: 259-266 (2005)). In breast cancer cells cultured in vitro, hCG has previously been shown to be tumoricidal as a single agent and to act as a radiosensitizer (Pond-Tor et al., Breast Cancer Res Treat., 72: 45-51 (2002)). In addition, in vitro studies using hCG have demonstrated that this growth factor can radiosensitize mammary carcinoma cells although no mechanistic analyses as to the mode of cell killing were performed (Salhab et al., Int J Fertil Womens Med. 50: 259-266 (2005)).

Of interest to the present invention are the disclosures of McMichael, U.S. Pat. Nos. 4,692,332 and 5,610,136 which relates to the use of equine chorionic gonadotropin and human chorionic gonadotropin (hCG) in combination with an immune enhancer such as a lysate of Staphylococcus aureus for treatment of malignant neoplasia and methods of treating benign prostatic hypertrophy by administration of a chorionic gonadotropin or a pharmaceutically active fragment or derivative thereof, respectively.

Also of interest to the present invention are the disclosures of Gallo et al., U.S. Pat. Nos. 6,805,882; 6,699,834; 6,699,656; 6,620,416; 6,596,688; 6,583,109; 6,319,504; 5,997,871; 5,968,513; 5,877,148; 5,677,275, which disclose the use of human chorionic gonadotropin for the treatment of various disorders including cancer, HIV, hematological disorders and wasting syndromes.

HMG-CoA (3-hydroxy-3-methylglutaryl-coenzyme A) reductase is the enzyme which catalyzes the rate limiting step of cholesterol biosynthesis. HMG-CoA reductase inhibitors, also known as statins, are molecules which inhibit the enzymatic activity of HMG-CoA reductase and have been used to treat patients suffering from hypercholesterolemia. The first such inhibitor (compactin or Mevastatin) was isolated in 1976 (Endo, A. et al., F.E. B.S. Lett., 72: 323-326, 1976) and since then many other natural and chemically modified versions of Mevastatin have been identified and developed for clinical use, including lovastatin, and Simvastatin. Statins are competitive inhibitors of 3-hydroxy-3-methylglutarlcoenzyme A (HMG-CoA) reductase, the enzyme that converts HMG-CoA to the cholesterol precursor mevalonic acid. Upon binding to the active site of HMG-CoA reducatase, statins alter the conformation of the enzyme, thereby preventing it from attaining a functional structure. The conformational change of the HMG-CoA reducatase active site makes statin drugs very effective and specific.

Several studies have reported that HMG-CoA reductase inhibitors have anti-angiogenic activity. For example, Feleszko et al., Int. J. Cancer, 81: 560-567 (1999) reported that treatment of a mouse model of tumor-cell induced angiogenesis with a combination of TNF-α and lovastatin produced a significant inhibition of tumor-induced blood-vessel formation whereas treatment with either TNF-α or lovastatin alone showed no angiostatic effects. Jones, M. K. et al., Am. J. Physiol., 276: G1345-GI1355 (1999) reported that mevastatin, an inhibitor of Ras activation, completely blocked the induction of VEGF (a potent angiogenic factor) expression in cultured primary endothelial cells. Kong, D. et al., Circulation, 100(18): 1-39, Abstract #194 (1999) reported that simvastatin exerted potent anti-angiogenic effects independent of its cholesterol lowering effects.

In addition to the studies suggesting that statins have anti-angiogenic effects certain retrospective studies have suggested that statins might reduce the risk of cancers of the colon, breast, lung, and prostate. Two recent studies show an anti-cancer effect from popular cholesterol-lowering drugs. One showed a reduction in prostate cancer recurrence and another laboratory study showed statins effective against bladder cancer cells (Moyad et al., Urology, 66:1150-1154, 2005; Kamat et al., 66:1209-1212, 2005).

Also of interest is the disclosure that agents containing lactone moieties including farnesyl transferase inhibitors (FTI), geranylgeranyltransferase inhibitors (GGTI) and statins such as lovastatin may have anticancer effects. Efuet et al., Cancer Res. 66, 1040-1051 (2006) suggests that such agents induce G₁ arrest by targeting the peoteasome. See also Santucci, et al., Cancer Control Vol. 10. No. 5, 384-387 (2003) which discloses the use of the FTI tipifamib in the treatment of multiple myeloma.

However, a review of 26 randomized, controlled trials (JAMA, 295:74-80, 2006) reports that statins have a neutral effect on cancer and cancer death risk in randomized controlled trials and that no type of cancer was affected by statin use and no subtype of statin affected the risk of cancer. The 26 clinical trials included in the meta-analysis enrolled more than 86,000 patients. They recorded more than 6,600 cancer diagnoses and more than 2,400 cancer deaths among those patients. The researchers found no statistically significant differences in the number of cancer diagnoses or deaths between patients who were randomly assigned to take statins and those who were not. Their findings were the same for all cancer types reviewed (breast, colon, and prostate cancer; respiratory and gastrointestinal cancers; and melanoma).

Prostate cancer is a disease of older males (>50 years). Upon initial presentation, the majority of patients present with tumors that are located within the prostate and tumor cells that are dependent upon androgen for growth and survival (Messing et al., Lancet Oncol. 7: 472-479 (2006) and Collette et al., Eur J Cancer 42: 1344-1350 (2006)). Primary forms of therapy for these patients include surgery and radiotherapy. However, if tumor cell clonogens survive in the prostate after therapy or were already present at local regional sites e.g. lymph nodes, tumors will eventually reoccur, and at present the primary mode of treating individuals with recurrent tumors is to ablate androgen signaling by use of a variety of agents e.g. Finasteride (Ryan et al., Curr Opin Oncol 16: 242-246 (2004) and Flores et al., Mini Rev Med Chem. 3: 225-237 (2003)). Androgen ablation therapy has the short-term effect of inducing tumor cell growth arrest and cell killing, with a parallel reduction in the plasma levels of prostate specific antigen (PSA), followed by a longer-term repopulation of androgen independent prostate cancer cells who have adapted their intracellular signaling pathways to survive, including increased expression of growth factor receptors e.g. ERBB1, insulin-like growth factor 1 receptor, and inactivation of tumor suppressor genes e.g. PTEN with parallel activation of PDK-1—AKT survival signaling (Sansal et al., J Clin Oncol. 22: 2954-2963 (2004); Roberts, Jr., Novartis Found Symp. 262: 193-199 (2004); and Barton et al., Urology 58: 114-122 (2001)). Specific inhibition of ERBB1 and/or phosphatidyl inositol 3 kinase (PI3K)—AKT signaling has been proposed by others as a therapeutic approach to suppress androgen-independent prostate cancer cell growth and enhance tumor cell radiosensitivity (e.g. Formento et al., Eur J Cancer; 40: 2837-2844 (2004)). As the development of androgen independent prostate cancer is invariably eventually fatal, the development of novel therapies against prostate cancer has clinical value.

Many groups have shown that the epidermal growth factor receptor (EGFR, also referred to as ERBB1 and HER1) is activated in response to irradiation of various carcinoma cell types (reviewed in Dent et al., Oncogene 22: 5885-5896 (2003) and Dent et al., Radiat Res. 59: 283-300 (2003)). Radiation exposure in the range 1-2 Gy, via activation of the ERBB1, can activate the ERK½ pathway to a level similar to that observed by physiologic, growth stimulatory, EGF concentrations (˜0.1 nM). Radiation-induced reactive oxygen species appear to play an important role in the activation of ERBB family receptors. The actions of ERBB receptor autocrine ligands have also been shown to play important roles in the activation of receptors after radiation exposure. For example, TGFα mediates secondary activation of the ERBB1 and the downstream ERK½ pathway after irradiation of several carcinoma cell lines, including androgen-independent prostate cancer cells (Hagan et al., Clin Cancer Res. 10: 5724-5731 (2004)). In this instance, radiation-induced activation of ERK½ promotes cleavage of pro-TGFα in the plasma membrane leading to growth factor release where it feeds back onto the irradiated cell: a stimulus response signaling loop (Shvartsman et al., Am J Physiol Cell Physiol 282: C545-559 (2002)). Growth factors e.g. IGF1 have also been shown to promote ERBB1 and ERK½ activation in tumor cells by this circuitous route through the actions of other paracrine ligands e.g. HB-EGF (El-Shewy et al., Mol Endocrinol 18: 2727-2739 (2004)). In isolated rat luteal membranes, ERBB1 signaling has been shown to uncouple hCG from cAMP production, and in intact rat granulose cells hCG actions were potentiated by EGF, suggesting that ERBB1 signaling and hCG signaling may interact in hCG responsive cell types (Hattori et al., J Mol Endocrinol 15: 283-291 (1995) and Lamm et al., Endocrinology; 140: 29-36 (1999)).

### SUMMARY OF THE INVENTION

The present invention relates to the discovery that the combination of chorionic gonadotropin with an HMG CoA reductase inhibitor such as lovastatin is unexpectedly efficacious in the treatment of cancer. Accordingly, the present invention provides methods of treating cancer in a subject comprising administering to a subject in need thereof a therapeutically active amount of a composition comprising chorionic gonadotropin or a therapeutically active fragment or analogue there in combination with an HMG CoA reductase inhibitor. In some embodiments, the subject is a mammalian subject. In a particular embodiment, the subject is a human subject.

In some embodiments, the chorionic gonadotropin is hCG or a therapeutically active fragment or analogue thereof. In a particular embodiment, the therapeutically active fragment is a beta subunit of hCG. In some embodiments, the HMG CoA reductase inhibitor is a statin selected from the group consisting of lovastatin, simvastatin, pravastatin, fluvastatin, atorvastatin, itavastatin, rosuvastatin and rivastatin. In a particular embodiment, the statin is lovastatin.

The methods of the present invention can be used to treat a wide variety of cancers. For example, the cancer to be treated is selected from the group consisting of myxoid and round cell carcinoma, Ewing's sarcoma, cancer metastases, including lymphatic metastases, squamous cell carcinoma, esophageal squamous cell carcinoma, oral carcinoma, multiple myeloma, lymphocytic leukemia, acute nonlymphocytic leukemia, chronic lymphocytic leukemia, chronic myelocytic leukemia, and hairy cell leukemia, effusion lymphomas, thymic lymphoma lung cancer, small cell carcinoma of the lungs, cutaneous T cell lymphoma, Hodgkin's lymphoma, non Hodgkin's lymphoma, cancer of the adrenal cortex, non-small cell lung cancers, breast cancer, stomach cancer, colon cancer, colorectal cancer, colorectal neoplasia, pancreatic cancer, liver cancer, bladder cancer, primary superficial bladder tumors, invasive transitional cell carcinoma of the bladder, muscle invasive bladder cancer, prostate cancer, ovarian carcinoma, primary peritoneal epithelial neoplasms, cervical carcinoma, uterine endometrial cancers, vaginal cancer, cancer of the vulva, uterine cancer, solid tumors in the ovarian follicle, testicular cancer, penile cancer, kidney cancer, renal cell carcinoma, brain cancer, neuroblastoma, astrocytic brain tumors, gliomas, metastatic tumor cell invasion in the central nervous system, osteomas, osteosarcomas, malignant melanoma, tumor progression of human skin keratinocytes, basal cell carcinoma, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma. In a particular embodiment, the cancer to be treated is prostate cancer.

In one embodiment, the method of treating cancer comprises administering to a subject in need thereof a composition comprising hCG in a dosage ranging from about 0.2 IU to about 1000 IU, or from about 1 IU to about 500 IU, or from about 10 IU to about 100 IU. Lovastatin can be administered in a dosage ranging from about 0.1 mg/kg to about 1000 mg/kg, or from about 10 mg/kg to about 500 mg/kg, or from about 20 mg/kg to about 200 mg/kg.

In another embodiment, the methods described herein further comprise the step of treating an aforementioned cancer with radiation after administration of the chorionic gonadotropin and HMG CoA reductase inhibitor composition. In some embodiments, the radiation is external beam X-ray radiation. In other embodiments, the radiation by means of brachytherapy.

In yet another embodiment, the compositions described herein further comprise one or more additional agents that inhibit either NF-kappa-B activity, PI-3-kinase activity or AKT activity. In some embodiments, the one or more additional agents inhibit NF-kappa-B activity, PI-3-kinase activity and AKT activity.

Exemplary agents that inhibit NF-kappa-B activity include, but are not limited to, 2-chloro-N-[3,5-di(trifluoromethyl)phenyl]-4-(trifluoromethyl)pyrimidine-5-carboxamide (also known as SP-100030), 3,4-dihydro-4,4-dimethyl-2H-1,2-benzoselenazine (also known as BXT-51072), declopramide (also known as Oxi-104), dehydroxymethylepoxyquinomycin (DHMEQ), 6-Amino-4-(4-phenoxyphenylethylamino) quinazoline (QNZ), BAY 11-7082, BAY 11-7085, Caffeic Acid Phenethylester (CAPE), Lactacystin, Helenalin, MG-132 (Z-Leu-Leu-Leu-H), IKK-NBD Peptide, IKK-NBD Control Peptide, NF-κB SN50, NF-κB SN50M, Parthenolide, SC-514, Triptolide (PG490), Wedelolactone and dexlipotam.

Exemplary agents that inhibit PI-3-kinase activity include, but are not limited to, 2-(4-Morpholinyl)-8-phenyl-4H-1-benzopyran-4-one hydrochloride (LY294002), wortmannin, and SF1126 (Semafore Pharmaceuticals).

Exemplary agents that inhibit AKT activity include, but are not limited to, KP372-1, SR13668, 1L-6-Hydroxymethyl-chiro-inositol 2-(R)-2-O-methyl-3-O-octadecylcarbonate, SH-5, SH-6 (Kozikowski, A. P., et al., J. Am. Chem. Soc., 125:1144, 2003), AKT inhibitor IV (Kau, T. R., et al., Cancer Cell 4:463, 2003), 9-Methoxy-2-methylellipticinium acetate (MMEA), Triciribine (Yang, L., et al., Cancer Res., 64:4394, 2004), AKT-in (Hiromura, M., et al., J. Biol. Chem., 279:53407, 2004), TAT-Akt-in (Hiromura et al, supra), 1,3-Dihydro-1-(1-((4-(6-phenyl-1H-imidazo[4,5-g]quinoxalin-7-yl)phenyl)methyl)-4-piperidinyl)-2H-benzimidazol-2-one (AKT ½; Barnett, S. F., et al., Biochem. J., 385:399, 2005; DeFeo-Jones, D., et al., Mol. Cancer Ther., 4:271, 2005; Zhao, Z., et al., Bioorg. Med. Chem. Lett., 15:905, 2005. Lindsley, C. W., et al., Bioorg. Med. Chem. Lett., 15:761, 2005), 10-(4′-(N-diethylamino)butyl)-2-chlorophenoxazine HCl (Thimmaiah, K. N., et al., J. Biol. Chem., 280:3192, 2005), (−)-Deguelin, and 3-Formylchromone thiosemicarbazone, Cu(II)Cl2 Complex (Barve, V., et al., J. Med. Chem. 49: 3800, 2006).

In another embodiment, the compositions described herein further comprise one or more geranylgeranyltransferase inhibitors (GGTI) and/or farnesyltransferase inhibitors (FTI). As used herein GGTI refers to inhibitors of the geranylgeranyltransferase enzyme which do not have HMG-CoA reductase activity and FTI refers to inhibitors of farnesyltransferase enzymes that do not have HMG-CoA reductase activity.

Exemplary GGTIs include, but are not limited to, GGTI-2133 (N-[[4-(Imidazol-4-yl)methylamino]-2-(1-naphthyl)benzoyl]leucine trifluoroacetate salt) GGTI-2166, GGTI-298 (N-[[4-(2-(R)-Amino-3-mercaptopropyl)amino]-2-naphthylbenzoyl]leucine methyl ester trifluoroacetate salt), and GGTI-286.

Exemplary FTIs include, but are not limited to SSH-66363, BMS-214662, BMS-214664, L778,123, L744,823 ((2S)-2-[[(2S)-2-[[(2S,3S)-2-[[(2R)-2-Amino-3-mercaptopropyl]amino]-3-methylpentyl]oxy]-1-oxo-3-phenylpropyl]amino]-4-(methylsulfonyl)-Butanoic acid 1-methylethyl ester) lonafamib, zarnestra, R115777 (tipifamib), Manumycin A from Streptomyces parvulus, α-hydroxyfarnesylphosphonic acid, N-[4-[2-(R)-Amino-3-mercaptopropyl]amino-2-phenylbenzoyl]methionine trifluoroacetate salt, and N-[4-[2(R)-Amino-3-mercaptopropyl]amino-2-phenylbenzoyl]methionine methyl ester trifluoroacetate salt.

The invention also provides pharmaceutical compositions for the treatment of cancer comprising a therapeutically effective amount of a composition comprising a chorionic gonadotropin or a therapeutically active fragment or analogue thereof in combination with an HMG CoA reductase inhibitor. In some embodiments, the chorionic gonadotropin is hCG or a therapeutically active fragment or analogue thereof. In a particular embodiment, the therapeutically active fragment is a beta subunit of hCG. In some embodiments, the HMG CoA reductase inhibitor is a statin selected from the group consisting of lovastatin, simvastatin, pravastatin, fluvastatin, atorvastatin, itavastatin, rosuvastatin and rivastatin. In a particular embodiment, the statin is lovastatin.

The pharmaceutical compositions of the invention can be used to treat a wide variety of cancers. For example, the cancer to be treated is selected from the group consisting of myxoid and round cell carcinoma, Ewing's sarcoma, cancer metastases, including lymphatic metastases, squamous cell carcinoma, esophageal squamous cell carcinoma, oral carcinoma, multiple myeloma, lymphocytic leukemia, acute nonlymphocytic leukemia, chronic lymphocytic leukemia, chronic myelocytic leukemia, and hairy cell leukemia, effusion lymphomas, thymic lymphoma lung cancer, small cell carcinoma of the lungs, cutaneous T cell lymphoma, Hodgkin's lymphoma, non Hodgkin's lymphoma, cancer of the adrenal cortex, non-small cell lung cancers, breast cancer, stomach cancer, colon cancer, colorectal cancer, colorectal neoplasia, pancreatic cancer, liver cancer, bladder cancer, primary superficial bladder tumors, invasive transitional cell carcinoma of the bladder, muscle invasive bladder cancer, prostate cancer, ovarian carcinoma, primary peritoneal epithelial neoplasms, cervical carcinoma, uterine endometrial cancers, vaginal cancer, cancer of the vulva, uterine cancer, solid tumors in the ovarian follicle, testicular cancer, penile cancer, kidney cancer, renal cell carcinoma, brain cancer, neuroblastoma, astrocytic brain tumors, gliomas, metastatic tumor cell invasion in the central nervous system, osteomas, osteosarcomas, malignant melanoma, tumor progression of human skin keratinocytes, basal cell carcinoma, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma. In a particular embodiment, the cancer to be treated is prostate cancer.

In one embodiment, the pharmaceutical compositions for treating cancer comprise administering to a subject in need thereof a composition comprising hCG in a dosage ranging from about 0.2 IU to about 1000 IU, or from about 5 IU to about 500 IU, or from about 10 IU to about 100 IU. Lovastatin can be administered in a dosage ranging from about 0.1 mg/kg to about 1000 mg/kg, or from about 10 mg/kg to about 500 mg/kg, or from about 20 mg/kg to about 200 mg/kg.

In another embodiment, the pharmaceutical compositions of the invention further comprise one or more geranylgeranyltransferase inhibitors (GGTI) and/or farnesyl transferase inhibitors (FTI).

The present invention also contemplates that the combination of chorionic gonadotropin with a geranylgeranyltransferase inhibitor (GGTI) and/or farnesyl transferase inhibitor (FTI) would be efficacious in the treatment of cancer. Accordingly, the present invention provides methods of treating cancer in a subject comprising administering to a subject in need thereof a therapeutically active amount of a composition comprising chorionic gonadotropin or a therapeutically active fragment or analogue there in combination with a geranylgeranyltransferase inhibitor (GGTI). Similarly, methods of treating cancer in a subject comprising administering to a subject in need thereof a therapeutically active amount of a composition comprising chorionic gonadotropin or a therapeutically active fragment or analogue there in combination with a farnesyl transferase inhibitor (FTI).

In some embodiments, the chorionic gonadotropin is hCG or a therapeutically active fragment or analogue thereof. In a particular embodiment, the therapeutically active fragment is a beta subunit of hCG.

In one embodiment, the method of treating cancer comprises administering to a subject in need thereof a composition comprising hCG in a dosage ranging from about 0.2 IU to about 1000 IU, or from about 5 IU to about 500 IU, or from about 10 IU to about 100 IU. GGTI can be administered in a dosage ranging from about 1 mg/m² to about 1000 mg/m², or from about 5 mg/m² to about 500 mg/m² or from about 10 mg/m² to about 100 mg/m². In another embodiment, the method of treating cancer comprises administering to a subject in need thereof a composition comprising FTI in a dosage ranging 1 mg/m² to about 1000 mg/m², or from about 5 mg/m² to about 500 mg/m² or from about 10 mg/m² to about 100 mg/m².

The invention also provides pharmaceutical compositions for the treatment of cancer comprising a therapeutically effective amount of a composition comprising a chorionic gonadotropin or a therapeutically active fragment or analogue thereof in combination with a GGTI and/or FTI. In some embodiments, the chorionic gonadotropin is hCG or a therapeutically active fragment or analogue thereof. In a particular embodiment, the therapeutically active fragment is a beta subunit of hCG.

In one embodiment, the pharmaceutical compositions for treating cancer comprise administering to a subject in need thereof a composition comprising hCG in a dosage ranging from about 0.2 IU to about 1000 IU, or from about 5 IU to about 500 IU, or from about 10 IU to about 100 IU. GGTI can be administered in a dosage ranging from about 1 mg/m² to about 1000 mg/m², or from about 5 mg/m² to about 500 mg/m² or from about 10 mg/m² to about 100 mg/m². In another embodiment, the method of treating cancer comprises administering to a subject in need thereof a composition comprising FTI in a dosage ranging 1 mg/m² to about 1000 mg/m², or from about 5 mg/m² to about 500 mg/m² or from about 10 mg/m² to about 100 mg/m².

The compositions of the invention can be administered by various routes that include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE FIGURES

FIGS. 1A-1C: Depict the results of apoptosis assays in LNCaP and PC-3 cells after treatment with hCG and radiation.

FIG. 1D: Depicts the results of an LNCaP cell proliferation assay after pretreatment with a caspase inhibitor followed by treatment with hCG and radiation.

FIG. 1E: Depicts the results of apoptosis assays in LNCaP cells after pretreatment with a caspase inhibitor followed by treatment with hCG and radiation.

FIG. 1F: Depicts the results of an LNCaP cell survival assay after treatment with hCG and radiation.

FIG. 2A-2D: Depict the results of apoptosis assays in LNCaP cells after pretreatment with an ERBB1 inhibitor, constitutively active MEK1 or AKT, dominant negative MEK1 or AKT, or JNK½ inhibitor peptide followed by treatment with hCG and radiation.

FIGS. 3A-3B: Depict the results of apoptosis assays in LNCaP cells after pretreatment with a PARP inhibitor or siRNA molecules to suppress PARP1 or AIF expression followed by treatment with hCG and radiation.

FIG. 3C: Depicts the results of an LNCaP cell survival assay after pretreatment with an ERBB1 small molecule inhibitor (AG1478) followed by treatment with hCG and radiation.

FIG. 4: Depicts the results of apoptosis assays in LNCaP cells after pretreatment with an ERBB1 small molecule inhibitor (AG1478) or a P3K inhibitor (LY294002) followed by treatment with hCG and radiation.

FIG. 5A: Depicts the results of apoptosis assays in LNCaP cells after treatment with hCG (1-2.0 U/ml) and lovastatin (0.1-0.6 μM).

FIG. 5B: Depicts the results of apoptosis assays after pretreatment with farnesyl pyrophosphate (FPP; 1 μM) or geranylgeranyl pyrophosphate (GGPP, 1 μM) followed by treatment with hCG and lovastatin.

FIG. 5C: Depicts the results of an LNCaP cell survival assay after treatment with hCG (1-2.0 mU/ml) and lovastatin (0.1-0.6 μM).

FIG. 6A: Depicts the results of apoptosis assays in 22RW1 cells after treatment with lovastatin (0.1-0.6 μM) followed by the combination hCG (2.0 mU/ml) and lovastatin (0.1-0.6 μM).

FIG. 6B: Depicts the results of apoptosis assays in PC-3 cells after treatment with lovastatin (0.1-0.6 μM) followed by the combination hCG (1 mU/ml) and lovastatin (0.1-1.0 μM).

FIG. 6C: Depicts the results of apoptosis assays in SKVO3 cells after treatment with lovastatin (0.1-0.6 μM) followed by the combination hCG (2.0 mU/ml) and lovastatin (0.1-1.0 μM).

FIGS. 7A-7B: Depict the results of apoptosis assays in LNCaP cells after pretreatment with constitutively active MEK1 or AKT, dominant negative MEK1 or AKT, dominant negative IkB or JNK½ inhibitor peptide followed by treatment with hCG and lovastatin.

FIGS. 8A-8B: Depict the results of apoptosis assays in LNCaP cells after pretreatment with a caspase inhibitor followed by treatment with hCG and lovastatin.

### DETAILED DESCRIPTION

The present invention relates to the discovery that the combination of a chorionic gonadotropin with an HMG CoA reductase inhibitor is unexpectedly efficacious in the treatment of cancer. Accordingly, the present invention provides methods of treating cancer in an individual comprising administering a therapeutically effective amount of a composition comprising hCG and a statin. Also, provided are method of treating cancer in an individual comprising treating said cancer with radiation after administration of the hCG and statin composition.

Chorionic gonadotropins which are useful in practice of the invention include human (hCG) and equine chorionic gonadotropins as well as biologically active subunits, fragments and analogues thereof. The β-subunit of hCG, and fragments and analogues thereof, are useful whether or not complexed to the α-subunit that hCG. It is further believed that the gonadotropins follicle stimulating hormone (FSH) and leutenizing hormone (LH) and biologically active fragments and analogues thereof can also be combined with the HMG CoA reductase inhibitors of the invention as well as with farnesyl and geranygeranyl transferase inhibitors of the invention to effect improved cancer therapies.

hCG and biologically active fragments and analogues thereof is available from several sources including commercially from Sigma Chemical Co. (St. Louis, Mo.). hCG is available in a form suitable for therapeutic use from Wyeth-Ayerst Laboratories (APL™, Philadelphia Pa.), Organon, Inc. (Pregnyl™, West Orange, N.J.), and Serono Laboratories, Inc. (Profasi™, Randolph Mass.). Once purified, partially or to homogeneity as desired, the hormones can then be used therapeutically.

It is recognized that amino acid residues in the hormone polypeptides may be replaced by other amino acid residues with similar chemical properties (e.g., charge or hydrophobicity). Because the substituted amino acids have similar properties, the substitutions do not change the functional properties of the polypeptides. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

### Identifying Biologically Active hCG Fragments

Hormone polypeptides that are effective against cancers include the intact hCG and dimers, the β-subunits of hCG and subfragments of the hCG β-subunits. Among the biologically active subfragments of β-hCG that are effective against cancer are β-hCG(109-145) (Stevens (1986) CIBA Foundation Symp. 19: 200-225) and β-hCG(109-119) (Lyer et al. (1992) Int. J. Peptide Prot. Res. 39: 137-192). These latter polypeptides are available commercially from Bachem Bioscience, Inc. (King of Prussia, Pa.), and β-hCG(109-145) is also available from Sigma Chemical Co.

So long as the polypeptide or subfragment retains biological activity, it can be used in the claimed cancer treatment methods. To determine whether a particular polypeptide is biologically active, cells that express the hLH-hCG receptor (LH-CG-R) are exposed to the polypeptide fragment being tested, after which the cells are assayed for biological effects that are indicative of hCG presence. One can conduct this assay using a mammalian cell line that naturally expresses the LH-CG-R. One such cell type that is useful for this assay is the MA-10 transformed murine Leydig cell line [Ascoli (1981) Endocrinology 108: 88-95]. The MA-10 cells are grown as described by Chen and Puett [(1991a) J. Biol. Chem. 266: 6904-6908; Chen and Puett (1991b) Biochemistry 30: 10171-10175; Chen et al. (1991) J. Biol. Chem. 266: 19357-19361].

As an alternative to cells that naturally express LH-CG-R, one can use cells that express LH-CG-R because the cells have been transfected with an expression vector that harbors the LH-CG-R gene or cDNA. A suitable expression vector and cell line, as well as the nucleotide sequence of the LH-CG-R cDNA, are described in McFarland et al. (1989) Science 245: 494-499.

LH-CG-R-expressing cells exposed to a biologically active fragment of hCG or hLH will have elevated concentrations of cyclic AMP (cAMP). cAMP assays are described in, for example, McFarland et al., supra., Ascoli et al. (1989) J. Biol. Chem. 264: 6674, and Segaloff and Ascoli (1981) J. Biol. Chem. 256: 11420. A biologically active hormone fragment will cause cells treated with 10 ng/ml of the fragment for 15 minutes at 37° C. to have cAMP levels at least about 1.5 times as great as cells not treated with the fragment.

Another commonly used assay is to determine whether cells treated with the polypeptide produce higher levels of progesterone than untreated cells. The hormone polypeptide is added at various concentrations to the cells in a suitable medium. After a four hour incubation at 37°, progesterone is measured by radioimmunoassay. Basal progesterone concentration for untreated MA-10 cells is typically less than 10 ng/ml, while cells incubated in the presence of a biologically active hormone polypeptide will typically produce at least 500 ng/ml progesterone (Huang et al. (1993) J. Biol. Chem. 268: 9311-9315).

An alternative assay to assess whether a hCG polypeptide or subfragment is biologically active is to determine whether the polypeptide binds to the cellular receptor for hCG and hLH. A suitable assay is described in Huang et al., supra.

### HMG-CoA Reductase Inhibitors

Suitable HMG-Co-A reductase inhibitors which are useful for practice of the invention include statins such as mevastatin and related compounds which are disclosed in U.S. Pat. No. 3,983,140; and lovastatin (mevinolin) and related compounds which are disclosed in U.S. Pat. No. 4,231,938. Keto analogs of mevinolin (lovastatin) are disclosed in European Patent Application No. 0,142,146 A2, and quinoline and pyridine derivatives are disclosed in U.S. Pat. Nos. 5,506,219 and 5,691,322. Other statins useful in the practice of the invention include pravastatin and related compounds which are disclosed in U.S. Pat. No. 4,346,227; simvastatin and related compounds which are disclosed in U.S. Pat. Nos. 4,448,784 and 4,450,171; fluvastatin and related compounds which are disclosed in U.S. Pat. No. 5,354,772; cerivastatin and related compounds which are disclosed in U.S. Pat. Nos. 5,006,530 and 5,177,080; atorvastatin and related compounds which are disclosed in U.S. Pat. Nos. 4,681,893; 5,273,995; 5,385,929 and 5,686,104; pitavastatin (nisvastatin (NK-104) or itavastatin) and related compounds which are disclosed in U.S. Pat. No. 5,011,930. Rosuvastatin (visastatin (ZD-4522)) and related compounds are disclosed in U.S. Pat. No. 5,260,440. Other possible HMG CoA reductase inhibitor molecules are described in U.S. Pat. Nos. 5,753,675; 4,613,610; 4,686,237; 4,647,576; and 4,499,289; and British patent no. GB 2205837 the disclosures of which are all incorporated by reference.

The patents cited in relation to statins or other agents identified herein describe how to make and use the statins/agents, as well as biochemically active homologs thereof, salts, pro-drugs, metabolites, and the like. Such patents are incorporated herein by reference in their entirety. Dosings for the statins also have been described in patent and trade literature (e.g., Physician's Desk Reference 2004, incorporated herein by reference) and by the manufacturers and clinical practitioners that prescribe them. Combination therapy using statin dosings similar to what is used when prescribing statins alone, or less, is specifically contemplated.

### Radiation Therapy

Radiotherapy, including but not limited to external beam and/or brachytherapy, is a primary mode of treatment for prostate cancer. One site of dose limiting toxicity for prostate cancer radiotherapy is the sigmoid colon, and a variety of techniques, including intensity modulated radiotherapy and brachytherapy have been used to target tumor and spare normal tissue (De Meerleer et al., Int. J. Radiat. Oncol. Biol. Phys., 60:777-787, 2004 and Morton, G., Clin. Oncol (R Coll. Radiol., 14:219-227, 2005). Thus, it is contemplated that chorionic gonadotropins such as hCG and HMG CoA reductase inhibitors lovastatin could be used to enhance the toxic effects of radiotherapy in cancer.

### Therapeutic and Prophylactic Methods

The present invention provides methods for treating cancer comprising administering to a subject in need a therapeutic composition comprising a chorionic gonadotropin and an HMG CoA reductase inhibitor in therapeutically effective amounts. According to a preferred aspect of the invention, the chorionic gonadotropin is hCG and the HMG CoA reductase inhibitor is a statin with lovastatin being particularly preferred. The combination of a chorionic gonadotropin with an HMG CoA reductase inhibitor serves as a particularly effective radiosensitizer and a further aspect of the invention relates to the administration of radiation to a subject in need of treatment of cancer after administration of the chorionic gonadotropin with an HMG CoA reductase inhibitor combination.

Exemplary cancers include treatment of adult and pediatric oncology, growth of solid tumors/malignancies, myxoid and round cell carcinoma, locally advanced tumors, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma lung cancer (including small cell carcinoma of the lungs, cutaneous T cell lymphoma, Hodgkin's lymphoma, non Hodgkin's lymphoma, cancer of the adrenal cortex, ACTH producing tumors, non-small cell lung cancers, breast cancer, including small cell carcinoma and ductal carcinoma), gastro intestinal cancers (including stomach cancer, colon cancer, colorectal cancer, and polyps associated with colorectal neoplasia), pancreatic cancer, liver cancer, urological cancers (including bladder cancer, such as primary superficial bladder tumors, invasive transitional cell carcinoma of the bladder, and muscle invasive bladder cancer), prostate cancer, malignancies of the female genital tract (including ovarian carcinoma, primary peritoneal epithelial neoplasms, cervical carcinoma, uterine endometrial cancers, vaginal cancer, cancer of the vulva, uterine cancer and solid tumors in the ovarian follicle), malignancies of the male genital tract (including testicular cancer and penile cancer), kidney cancer (including renal cell carcinoma, brain cancer (including intrinsic brain tumors, neuroblastoma, astrocytic brain tumors, gliomas, and metastatic tumor cell invasion in the central nervous system), bone cancers (including osteomas and osteosarcomas), skin cancers (including malignant melanoma, tumor progression of human skin keratinocytes, basal cell carcinoma, and squamous cell cancer), thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

### Testing the Efficacy of the Therapeutic Composition

The clinician can test the efficacy of the therapeutic composition against a particular tumor type, either in vitro or in vivo. For in vitro tests, cells derived from the tumor are grown in the presence or absence of the therapeutic composition and the effect of the therapeutic composition is determined. One commonly utilized assay for tumor cell growth is the methylcellulose assay (Lunardi-Iskandar et al. (1985) Clin. Exp. Immunol. 60: 285-293). The cells are plated in medium containing methylcellulose, which prevents non-tumor cells from undergoing mitosis and forming colonies. The hCG will prevent tumor cells from forming as many colonies as untreated cells. Another means for measuring the inhibitory effect of the hCG polypeptides is by measuring the rate of incorporation of radiolabelled metabolites such as tritiated thymidine.

The growth of a cell line is said to be “inhibited” by therapeutic composition of the invention if, when assayed by means such as radioisotope incorporation into the cells, the treated cells proliferate at a rate that is less than about 80% of the proliferation rate of untreated control cells, and preferably less than about 70% of the untreated cell proliferation rate. More preferably, the growth rate is inhibited by at least 50%. If growth is assayed by a means such as plating in methylcellulose, the growth of a cell line is said to be “inhibited” if the treated cells give rise to less than about 80% of the number of colonies that grow from a like number of untreated cells. Preferably, the number of colonies from treated cells is less than about 70% of the number from untreated cells. More preferably, the number of colonies is decreased by at least 50%.

In addition to, or instead of, testing a therapeutic composition for efficacy against a particular tumor cell type in vitro, the clinician can test the therapeutic composition in vivo. For in vivo tests, cells derived from the tumor type are injected into laboratory animals such as immunodeficient mice. Typically, either the laboratory animals or the cells have been pre-treated with the hormone. The animals are then monitored to determine whether tumors arise at the site of injection, or elsewhere in the animal.

### Therapeutic Compositions and Routes of Administration

The invention provides methods of treatment and prevention by administration to a subject in need of such treatment of a therapeutically or prophylactically effective amount of a composition comprising chorionic gonadotropin and a statin. The subject is preferably an animal, including, but not limited to, animals such as monkeys, cows, pigs, horses, chickens, cats, dogs, etc., and is preferably a mammal. In some embodiments the subject is human.

The present invention also provides pharmaceutical compositions. Such compositions comprise a therapeutically effective amount of a Therapeutic, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the Therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In one embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water or saline. Where the composition is administered by injection, an ampule of sterile water for injection or saline can be provided so that the ingredients may be mixed prior to administration.

The amount of the therapeutic composition which will be effective in the treatment of a particular disorder or condition will depend on the nature of the disorder or condition, and can be determined by standard clinical techniques. An effective amount is a dosage of the therapeutic composition sufficient to provide a medically desirable result. In general, a therapeutically effective amount means that amount necessary to delay the onset of, inhibit the progression of, or halt altogether the particular condition being treated. In addition, in vivo and/or in vitro assays may optionally be employed to help predict optimal dosage ranges. The precise dose to be employed in the formulation will also depend on the route of administration, and the seriousness of the disease or disorder, and should be decided according to the judgment of the practitioner and each patient's circumstances:

For example, suitable doses of a chorionic gonadotropin for treatment or prevention of cancer include, but are not limited to, 0.2 to 1000 International units per day. Suitable doses of an HMG CoA reductase inhibitor (e.g., lovastatin) vary from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days. Suitable dosages for each of the components of the therapeutic composition may be determined empirically.

The therapeutic composition administration routes include, but are not limited to, intradermal, intramuscular, intraperitoneal, intravenous, subcutaneous, intranasal, epidural, and oral routes. The compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In one embodiment, it may be desirable to administer the pharmaceutical compositions of the invention locally to the area in need of treatment; this may be achieved, for example and not by way of limitation, by topical application, by injection, by means of a catheter, by means of a suppository, or by means of an implant, said implant being of a porous, non-porous, or gelatinous material, including membranes, such as sialastic membranes, or fibers.

In another embodiment, the therapeutic composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.)

In yet another embodiment, the therapeutic composition can be delivered in a controlled release system. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, thus requiring only a fraction of the systemic dose (see, e.g., Goodson, in Medical Applications of Controlled Release, supra, vol. 2, pp. 115-138 (1984)). Other controlled release systems are discussed in the review by Langer (Science 249:1527-1533 (1990)).

The invention also provides a pharmaceutical pack or kit comprising one or more containers filled with one or more of the ingredients of the therapeutic compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Other aspects, and advantages of the present invention will be understood upon consideration of the following illustrative examples, which are not intended to be limiting in any way.

### EXAMPLE 1

### Treatment of PC3 and LNCaP Cells with hCG Enhances their Radiosensitivity

This example demonstrates the radiosensitizing effects of hCG on human prostate cancer cells.

Cell culture. Human prostate cells (PC3 and LCNaP) were purchased from the ATCC were cultured at 37° C. (5% (v/v CO₂) in vitro using RPMI supplemented with 10% (v/v) fetal calf serum.

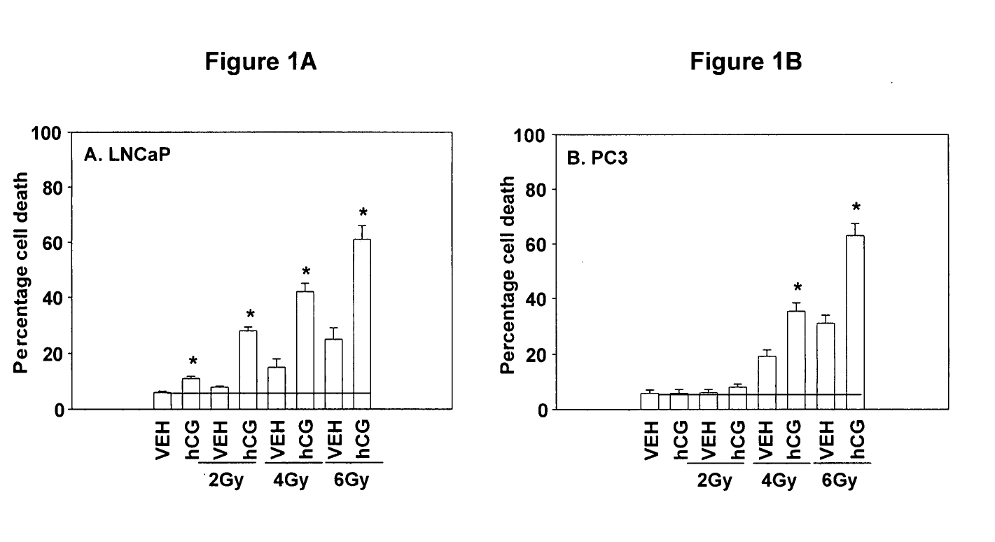

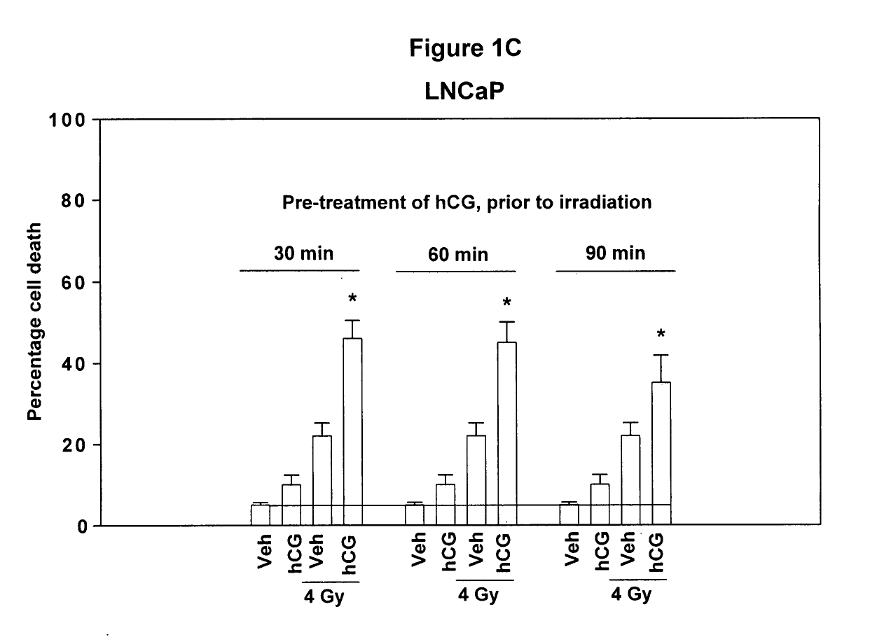

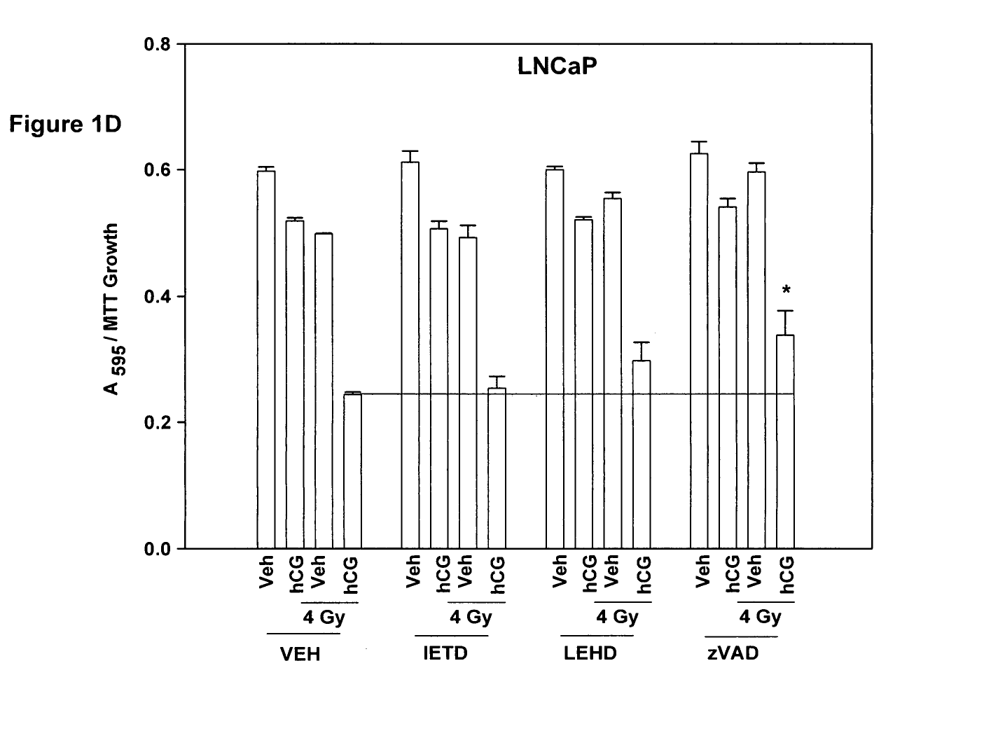

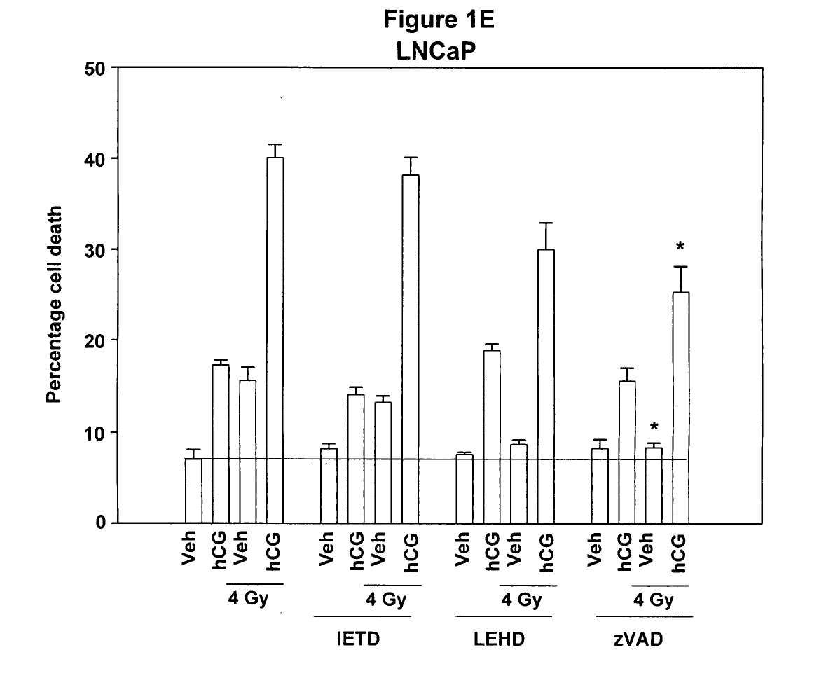

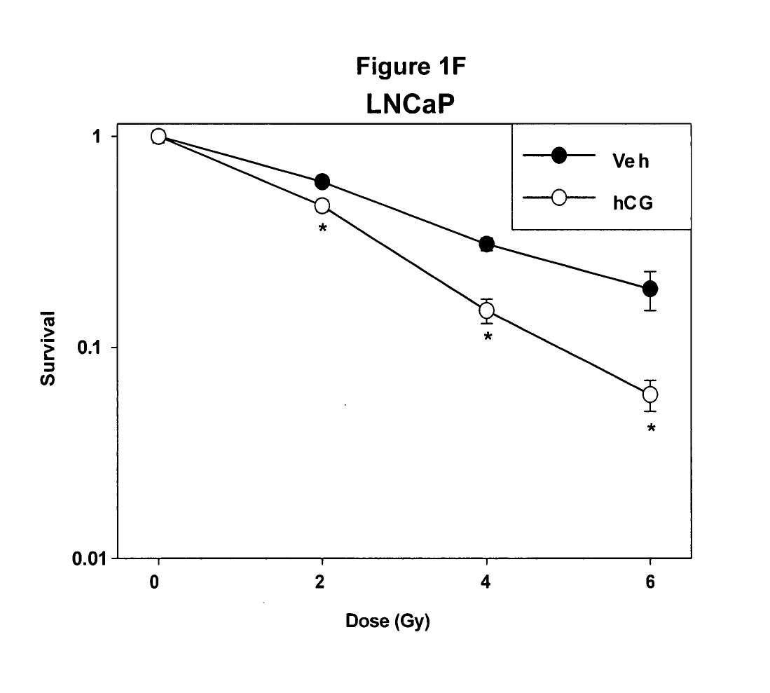

Apoptosis assay in PC3 cells (Treatment of hCG and radiation). PC3 cells were cultured as described above. 24 hours after plating, the cells were treated with hCG (ProSpec-Tany) (2 mU/ml) 30 minutes prior to radiation exposure (0-6 Gy). The cells were isolated by trypsinization 96 hours after irradiation. The attached and floating cells were combined, and cell viability was determined by trypan blue exclusion assays using a light microscope. Results indicated that the administration of hCG in combination with radiation promoted apoptosis at a greater percentage than the administration of hCG alone. The administration of hCG in combination with radiation (6 Gy) promoted the highest percentage of apoptosis. See FIG. 1B.

Apoptosis assay in LNCaP cells (Treatment of hCG and radiation). LNCaP cells were cultured as described above. 24 hours after plating, cells were treated with hCG (2 mU/ml) 30, 60 and 90 minutes prior to radiation exposure (4 Gy). The cells were isolated by trypsinization 96 hours after irradiation. The attached and floating cells were combined, and cell viability was determined by trypan blue exclusion assays using a light microscope (*p<0.05 value greater than corresponding vehicle/irradiated+vehicle value; n=3 per data point ±SEM from 3 studies). Results indicated that the administration of hCG in combination with radiation promoted apoptosis at a greater percentage than the administration of hCG alone. The administration of hCG in combination with radiation (6Gy) promoted the highest percentage of apoptosis. See FIGS. 1A and 1C.

Bioassay for cell growth and apoptosis assay (Pretreatment with a caspase inhibitor followed by hCG and radiation treatment). LNCaP cells were cultured as described above. Twenty four hours after plating, the cells were pre-treated (30 minutes) with either the caspase 8 inhibitor IETD (50 μM); the caspase 9 inhibitor LEHD (50 μM); the pan-caspase inhibitor zVAD (50 μM) followed by treatment with hCG (2 mU/ml) 30 minutes prior to radiation exposure (4 Gy). Caspase inhibitors were re-supplemented every 24 hours. After 96 hours of culture, cell viability numbers were determined MTT assays using a plate reader. The cells were isolated 96 hours after irradiation by trypsinization, the attached and floating cells combined, and cell viability determine by trypan blue exclusion assays using a light microscope. Results indicated that pretreatment with a caspase inhibitor prior to hCG and radiation treatment had little effect on the radiosensitization of hCG and promoted decreased cell growth in the MTT growth assay and promoted a higher percentage of apoptosis when compared to the controls. See FIGS. 1D-1E.

Bioassay for cell survival. LNCaP cells were cultured as described above. Twenty four hours after plating (250-2,500 cells/well of a 6 well plate), cells were treated with hCG (2 mU/ml) 30 min prior to radiation exposure (0-6 Gy). Ninety six hours after irradiation, media containing hCG was removed and fresh media lacking hCG added. Colonies were permitted to form over the following 28 days after which the media was removed, the cells fixed and stained with crystal violet. Groups of >50 cells were considered to be colonies. Results indicated that treatment with hCG and radiation promoted a decrease in cell survival when compared to the control. See FIG. 1F.

Results. Treatment of LNCaP and PC-3 cells with low concentrations of hCG (2 mU/ml) caused a modest amount of cell killing as a single agent within 96 hours, which was enhanced in a dose dependent fashion following exposure to ionizing radiation. An assessment was then made of the length of pre-treatment time with hCG required to cause radiosensitization. Pre-treatment of LNCaP and PC-3 cells with hCG for 12 hours or 6 hours did not significantly radiosensitize prostate carcinoma cells whereas pretreatment with hCG for 30-90 min enhanced the lethality of radiation (FIG. 1C).

In LNCaP cells, the pan-caspase inhibitor zVAD and to a lesser extent the caspase 9 inhibitor LEHD partially protected cells from the toxicity of ionizing radiation in MTT assays. Radiation exposure and hCG interacted to cause a greater than additive increase in cell death that was modestly blunted by zVAD and to a lesser extent by LEHD (FIG. 1E). Over-expression of dominant negative caspase 9 did not recapitulate the effects of LEHD in trypan blue assays, collectively arguing that the intrinsic death pathway was playing a modest role in the observed killing effect. In agreement with our short term cell killing analyses, exposure to hCG radiosensitized LNCaP and PC-3 cells in colony formation assays.

### EXAMPLE 2

### Paracrine Activation of ERBB1 in LNCaP Cells after Treatment with hCG

The following experiments demonstrate that hCG activates ERBB1 expression.

Cell culture. LNCaP cells were purchased from the ATCC were cultured at 37° C. (5% (v/v CO₂) in vitro using RPMI supplemented with 10% (v/v) fetal calf serum.

Colony formation assays. Cells for in vitro colony formation assays were plated at 250-4000 cells per well in sextupilcate and for in vitro assays 14 hours after plating were treated with either Vehicle (DMSO), AG1478 or PD184352 as indicated for 48 hours followed by drug removal. 10-14 days after exposure, plates were washed in PBS, fixed with methanol and stained with a filtered solution of crystal violet (5% w/v). After washing with tap water, the colonies were counted both manually (by eye) and digitally using a ColCount™ plate reader (Oxford Optronics, Oxford, England). Data presented is the arithmetic mean (±SEM) from both counting methods from multiple studies. Colony formation was defined as a colony of 50 cells or greater.

SDS-PAGE and Immunoblotting. The cells were plated at 5×10⁵ cells/cm² and treated with drugs at the indicated concentrations and after the indicated time of treatment, lysed in whole-cell lysis buffer (0.5 M Tris-HCl, pH 6.8, 2% SDS, 10% glycerol, 1% β-mercaptoethanol, 0.02% bromophenol blue), and the samples were boiled for 30 minutes. The boiled samples were loaded onto 10-14% SDS-PAGE and electrophoresis was run overnight. Proteins were electrophoretically transferred onto 0.22 μm nitrocellulose, and immunoblotted with various primary antibodies against different proteins. All immunoblots were visualized by enhanced chemiluminescence (ECL).

Total ERK2 and/or ERBB1 Protein Expression Assays.

(1) LNCaP cells were cultured as described above. 24 hours after plating, cells were treated with hCG (2 mU/ml) or vehicle (VEH, PBS). 0-6 hours after treatment, the cells were isolated, lysed in SDS PAGE sample buffer and equal protein concentrations subjected to SDS PAGE followed by immunoblotting to determine the phosphorylation status (activity status) of ERK½, JNK½, AKT (S473) and the expression level of ERK2 protein. Results indicated that treatment of the LNCaP cells with hCG promoted activation of ERK½ and to a lesser extent activation of JNK½ within 6 hours, but did not significantly alter the high basal level of AKT activity in these cells, and did not promote activation of the p38 MAPK pathway.

(2) LNCaP cells were cultured as described above. 12 hours after plating, the cells were treated with vehicle (VEH, PBS) or pertussis toxin (300 ng/ml). 23 hours after plating cells were treated with vehicle (VEH, DMSO) or the ERBB1 inhibitor AG1478 (1 μM). 24 hours after plating, cells were treated with hCG (2 mU/ml) or vehicle (VEH, PBS). Cells were isolated 30 min after hCG treatment, lysed in SDS PAGE sample buffer and equal protein concentrations subjected to SDS PAGE followed by immunoblotting to determine the phosphorylation status (activity status) of ERK½ and ERBB1 Y1173 and the expression level of ERK2 protein. Results indicated that pre-treatment of the LNCaP cells with a specific inhibitor of Gai subunit function, pertussis toxin, suppressed hCG-induced activation of ERK½ indicating that hCG activated this pathway in LNCaP cells in a Gai-dependent manner. Furthermore, treatment of LNCaP cells with the tyrphostin AG1478 or expression of dominant negative ERBB1-CD533 also blocked hCG-induced ERK½ activation which indicates that ERBB1 is a key player in hCG-mediated activation of ERK½. Further, treatment of LNCaP cells with hCG activated ERBB1, as judged by increased phosphorylation of ERBB1 Y1173, an effect that was blocked by the ERBB1 specific tyrphostin AG1478 and by treatment with pertussis toxin.

(3) LNCaP cells were cultured as described above. 24 hours after plating, the cells were treated with hCG (2 mU/ml) or its vehicle (VEH, PBS). After hCG treatment (0-180 min), the cells were isolated, lysed in SDS PAGE sample buffer and equal protein concentrations subjected to SDS PAGE followed by immunoblotting to determine the phosphorylation status (activity status) of ERBB1 Y1068/Y1173/Y992/Y845 and the expression level of ERK2 and ERBB1 protein. Results indicated that treatment of the LNCaP cells with hCG promoted prolonged phosphorylation of ERBB1 at Y1173 and Y1068, transient phosphorylation at Y845, and little detectable phosphorylation at Y992.

(4) LNCaP cells were cultured as described above. Twenty four hours after plating cells were pretreated with vehicle (VEH, DMSO), the MEK½ inhibitor PD184352 (1 μM), or the metalloprotease inhibitor GM6001 (1 μM), and then treated 30 minutes afterwards with hCG (2 mU/ml) or its vehicle (VEH, PBS). Thirty minutes after hCG treatment, cells were irradiated (4 Gy). Thirty minutes after irradiation the cells were isolated, lysed in SDS PAGE sample buffer and equal protein concentrations subjected to SDS PAGE followed by immunoblotting to determine the phosphorylation status (activity status) of ERK½ and ERBB1 Y1068 and the expression levels of ERBB1 and ERK2 proteins. Results indicated an increase in ERBB1 Y1173 phosphorylation induced by hCG, 30 min after treatment. This increase was further enhanced by radiation exposure and was abolished by incubation of cells with the specific MEK½ inhibitor PD184352. The MEK½ inhibitor treatment did not alter radiation-induced ERBB1 Y1173 phosphorylation at this time point. Finally, the cells were incubated with the metalloprotease inhibitor GM6001 and exposed to hCG demonstrated both abolisged ERBB1 Y1173 phosphorylation and ERK½ activation 30 min after hCG treatment.

Results. As inhibition of MEK½ blocked hCG-induced phosphorylation of ERBB1 Y1173, the effect of hCG on ERBB1 activation via a paracrine mechanism was studied. We next attempted to determine the paracrine factor mediating hCG-induced activation of ERBB1. We noted that LNCaP cells do not express high levels of heregulin, TGFa or HB-EGF, three well characterized paracrine ligands that bind to ERBB family receptors. However, inhibition of heregulin, TGFa or HB-EGF function by use of neutralizing antibodies did not alter ERBB1 Y1173 or ERK½ phosphorylation in LNCaP cells after hCG treatment. Thus, the results indicate that hCG causes ERK½ activation via a Gai-dependent mechanism which in turn promotes activation of a metalloprotease, leading to cleavage of a paracrine ligand which promotes prolonged activation of ERBB1 and of ERK½ activity.

### EXAMPLE 3

### Inhibition of ERBB1 and Activation of AKT and MEK1 Suppress the Radiosensitizing Effects of hCG in LNCaP Cells

This example demonstrates that the inhibition of ERBB1 suppresses the radiosentitizing effects of hCG.

Cell culture. LNCaP cells were purchased from the ATCC were cultured at 37° C. (5% (v/v CO₂) in vitro using RPMI supplemented with 10% (v/v) fetal calf serum.

Recombinant adeno viral vectors, generation and infection in vitro. Recombinant adenoviruses were generated to express constitutively activated and dominant negative AKT and MEK1 proteins, dominant negative ERBB1 (COOH-terminal 533 amino acid deletion; CD533), dominant negative JNK1 as described previously (Hagan et al., Clin Cancer Res. 10: 5724-5731, 2004; Caron et al., Mol Cancer Ther. 4: 257-270, 2005; Caron et al., Mol Cancer Ther. 4: 243-255, 2005; and Yacoub et al., Mol Pharmacol 70: 589-603, 2006). LCNaP cells were infected with these adenoviruses at an approximate multiplicity of infection (m.o.i.) of 25. Cells were further incubated for 24 hours to ensure adequate expression of transduced gene products prior to any drug exposures/assays.

Colony formation assays. Cells for in vitro colony formation assays were plated at 250-4000 cells per well in sextupilcate and for in vitro assays 14 hours after plating were treated with either Vehicle (DMSO), AG1478 or PD184352 or the drug combinations as indicated for 48 hours followed by drug removal. 10-14 days after exposure, plates were washed in PBS, fixed with methanol and stained with a filtered solution of crystal violet (5% w/v). After washing with tap water, the colonies were counted both manually (by eye) and digitally using a ColCount™ plate reader (Oxford Optronics, Oxford, England). Data presented is the arithmetic mean (±SEM) from both counting methods from multiple studies. Colony formation was defined as a colony of 50 cells or greater.

SDS-PAGE and Immunoblotting. The cells were plated at 5×10⁵ cells/cm² and treated with drugs at the indicated concentrations and after the indicated time of treatment, lysed in whole-cell lysis buffer (0.5 M Tris-HCl, pH 6.8, 2% SDS, 10% glycerol, 1% β-mercaptoethanol, 0.02% bromophenol blue), and the samples were boiled for 30 minutes. The boiled samples were loaded onto 10-14% SDS-PAGE and electrophoresis was run overnight. Proteins were electrophoretically transferred onto 0.22 μm nitrocellulose, and immunoblotted with various primary antibodies against different proteins. All immunoblots were visualized by enhanced chemiluminescence (ECL).

Apoptosis Assays.

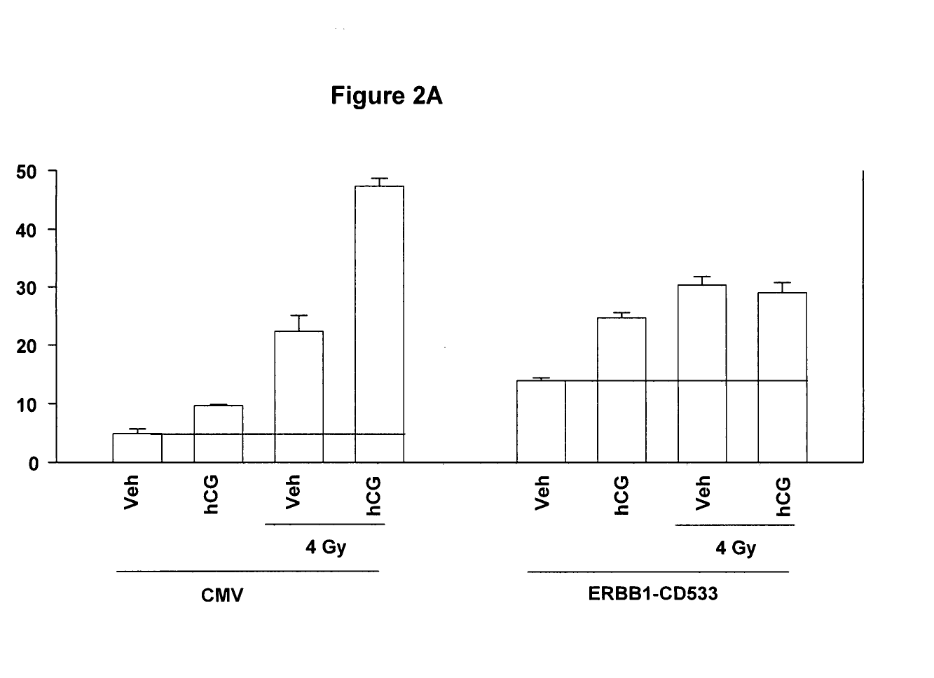

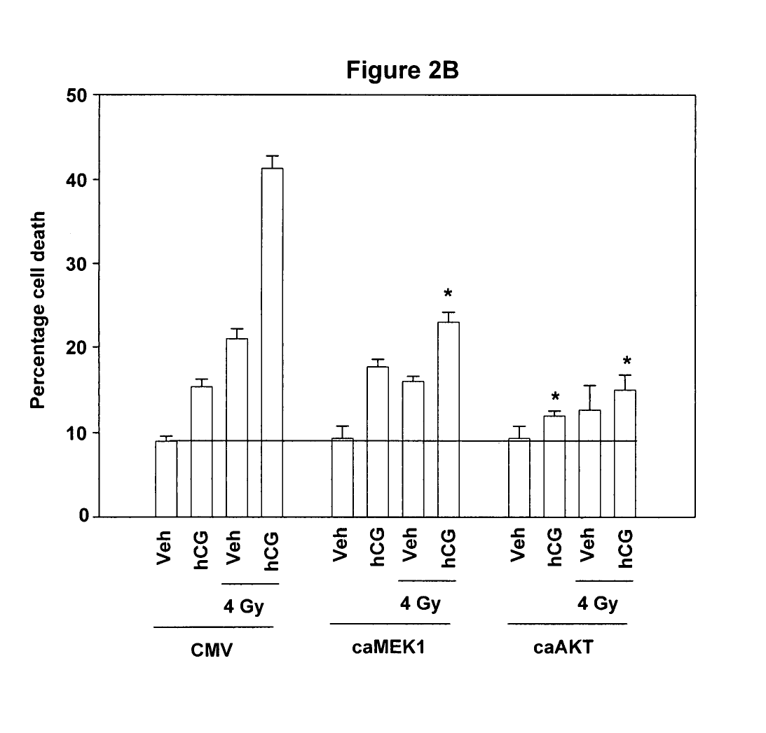

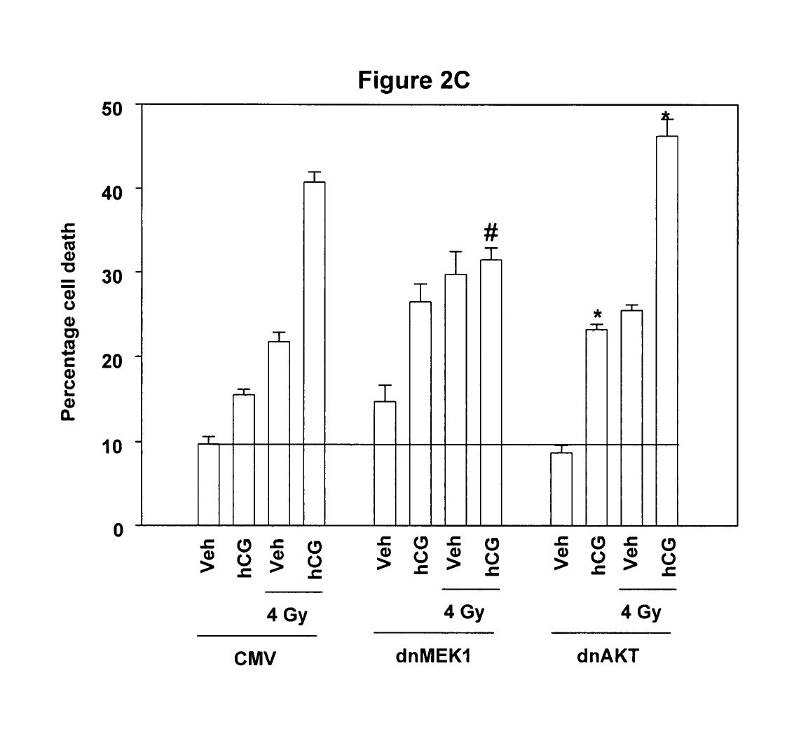

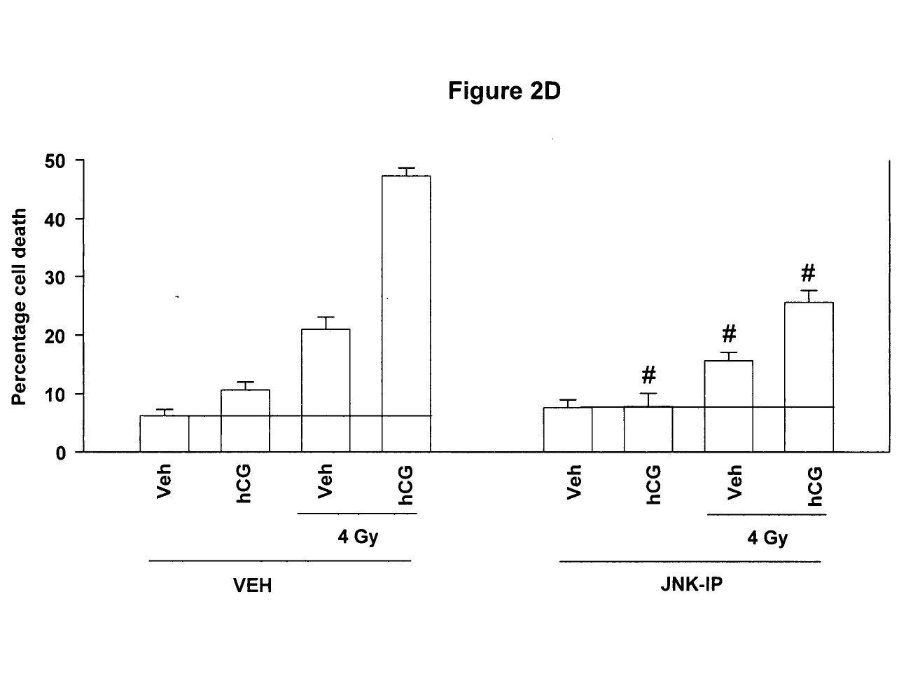

(1) LNCaP cells were cultured as described above. Twelve hours after plating, cells were infected with either a control, an empty vector virus (CMV), with a virus to express dominant negative ERBB1 (CD533), express constitutively active (ca) MEK1 or to express constitutively active AKT, dominant negative MEK1, or to express dominant negative (dn) AKT at a multiplicity of infection at a multiplicity of infection (m.o.i.) of 25. Twenty four hours after infection, cells were treated with hCG (2 mU/ml) or vehicle (VEH, PBS), and 30 minutes later irradiated (4 Gy). Cells were isolated 96 hours after irradiation by trypsinization, the attached and floating cells combined, and cell viability determine by trypan blue exclusion assays using a light microscope. Results indicated that expression of dominant negative ERBB1 (CD533) suppressed the enhanced lethality of hCG and radiation (FIG. 2A). Expression of activated MEK1 did not alter hCG the levels of induced cell death but suppressed the lethality of hCG and radiation exposure, whereas expression of activated AKT suppressed the lethality of hCG, radiation and hCG and radiation exposure (FIG. 2B). Expression of dominant negative AKT enhanced hCG lethality and further enhanced the lethality of hCG and radiation exposure (FIG. 2C). Expression of dominant negative MEK1, however, did not alter the lethality of hCG irradiation and modestly suppressed the lethality of hCG and radiation exposure (FIG. 2C)

(2) LNCaP cells were cultured as described above. Twenty four hours after plating cells were pre-treated with either the control, vehicle (VEH, DMSO), or the JNK½ inhibitor peptide (JNK-IP, 10 μM), and then treated 30 minutes afterwards with hCG (2 mU/ml) or its vehicle (VEH, PBS). Thirty minutes after hCG treatment, cells were irradiated (4 Gy). Cells were isolated 96 h after irradiation by trypsinization, the attached and floating cells combined, and cell viability determine by trypan blue exclusion assays using a light microscope. Results indicated that inhibition of JNK½ signaling suppressed the lethality of hCG, radiation and combined exposure to both hCG and radiation (FIG. 2D).

Results. Collectively, the findings in this example indicate that activation of ERBB1 is causal in the lethal interaction between hCG and radiation exposure, that a portion of the cell death signal is mediated by the JNK½ pathway, and that PI3K-AKT signaling, rather than MEK½-ERK½ signaling, plays a major role in protecting LNCaP cells from hCG toxicity.

### EXAMPLE 4

### ERBB1-Dependent PARP Activation is Causal in the Radiosensitizing Effects of hCG in LNCaP Cells

This example demonstrates that hCG and radiation exposure interact in an ERBB1-dependent manner to promote high levels of PARP activation, which is responsible for LNCaP cell radiosensitization by hCG.

Cell culture. LNCaP cells were purchased from the ATCC were cultured at 37° C. (5% (v/v CO₂) in vitro using RPMI supplemented with 10% (v/v) fetal calf serum.

Recombinant adeno viral vectors, generation and infection in vitro. Recombinant adenoviruses were generated to express constitutively activated and dominant negative AKT and MEK1 proteins, dominant negative ERBB1 (COOH-terminal 533 amino acid deletion; CD533), dominant negative JNK1 (Hagan et al., Clin Cancer Res. 10: 5724-5731, 2004; Caron et al., Mol Cancer Ther. 4: 257-270, 2005; Caron et al., Mol Cancer Ther. 4: 243-255, 2005; and Yacoub et al., Mol Pharmacol 70: 589-603, 2006). LCNaP cells were infected with these adenoviruses at an approximate multiplicity of infection (m.o.i.) of 25. Cells were further incubated for 24 hours to ensure adequate expression of transduced gene products prior to any drug exposures/assays.

SDS-PAGE and Immunoblotting. The cells were plated at 5×10⁵ cells/cm² and treated with drugs at the indicated concentrations and after the indicated time of treatment, lysed in whole-cell lysis buffer (0.5 M Tris-HCl, pH 6.8, 2% SDS, 10% glycerol, 1% β-mercaptoethanol, 0.02% bromophenol blue), and the samples were boiled for 30 minutes. The boiled samples were loaded onto 10-14% SDS-PAGE and electrophoresis was run overnight. Proteins were electrophoretically transferred onto 0.22 μm nitrocellulose, and immunoblotted with various primary antibodies against different proteins. All immunoblots were visualized by enhanced chemiluminescence (ECL).

Transfection of cells with siRNA molecules to suppress Apoptosis Inducing Factor (AIF) and Poly (ADP-ribose) Polymerase (PARP) expression. Cells were plated as described above and were transfected after 24 hours. RNA interference, or gene silencing for down-regulating the expression of PARP and AIF, was performed using validated target sequences designed by Ambion, Inc. (Austin, Tex.). For transfection, 10 nM of the annealed siRNA targeting PARP or AIF, the positive sense control doubled stranded siRNA targeting GAPDH or the negative control (a “scrambled” sequence with no significant homology to any known gene sequences from mouse, rat or human cell lines) were used. The small RNA sequences were transfected by electroporation at 600 V during 60 μsec. The cells were treated with drugs, as noted above, after 48 or 72 hours of transfection.

PARP1 Activation and Expression Following Treatment with hCG and Radiation. LNCaP cells were cultured as described above. Twenty four hours after plating, the cells were treated with hCG (2 mU/ml) or vehicle (VEH, PBS) followed 30 minutes later by irradiation (4 Gy). Thirty minutes after irradiation, cells were isolated, lysed in SDS PAGE sample buffer and equal protein concentrations subjected to SDS PAGE followed by immunoblotting to determine the ADP-ribosylation status (activity status) of PARP1 and the expression level of total PARP1 and β-actin protein levels. Results indicated that radiation and hCG acted in a greater than additive fashion to cause activation of poly ADP ribosyl polymerase (PARP) as judged by PARP1 ADP-ribosylation (Schreiber et al., Nat. Rev. Mol. Cell. Biol., 7:517-528, 2006).

PARP1 Activation and Expression Following Treatment with an ERBB1 Inhibitor, hCG and Radiation. LNCaP cells were cultured as described above. Twelve hours after plating, the cells were infected with either empty vector virus (CMV) or with a virus to express dominant negative ERBB1 (CD533) at a multiplicity of infection (m.o.i.) of 25. Twenty four hours after plating or 24 h after infection, cells were treated with hCG (2 mU/ml) or vehicle (VEH, PBS), and after 30 minutes were irradiated (4 Gy). Treatment with AG1478 or vehicle (VEH, DMSO) was made either 5 minutes after or 30 minutes before radiation. Thirty minutes after irradiation, cells were isolated, lysed in SDS PAGE sample buffer and equal protein concentrations subjected to SDS PAGE followed by immunoblotting to determine the PARP1 ADP-ribosylation status (activity status) and the expression level of total PARP1 and β-actin protein levels. Results indicated that inhibition of ERBB1 using either the small molecule inhibitor AG1478 or expression of dominant negative ERBB1 (CD533) abolished the profound activation of PARP caused by combined hCG and radiation treatment.

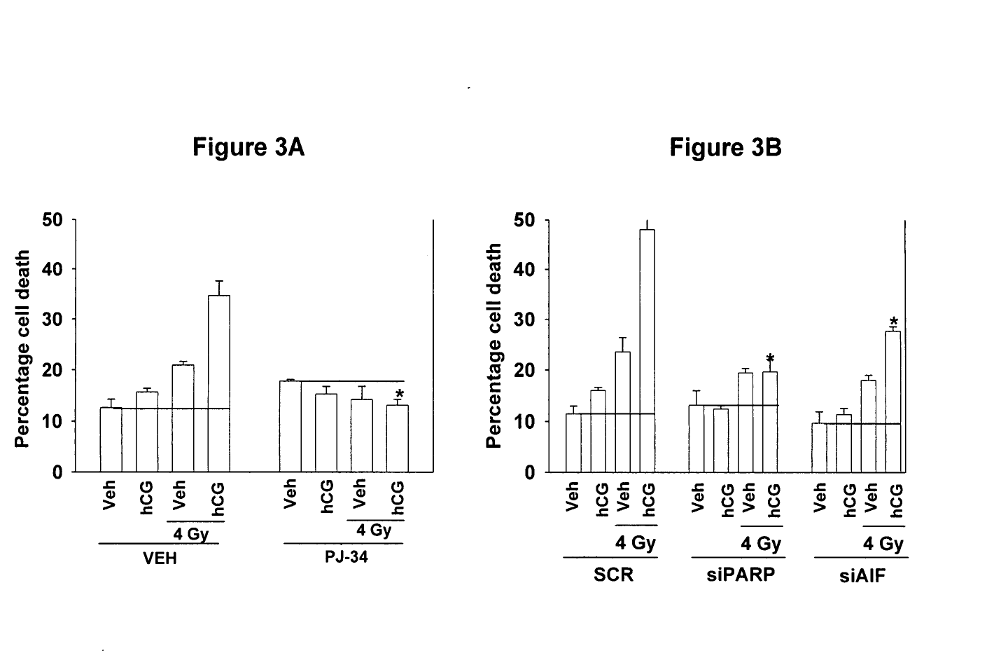

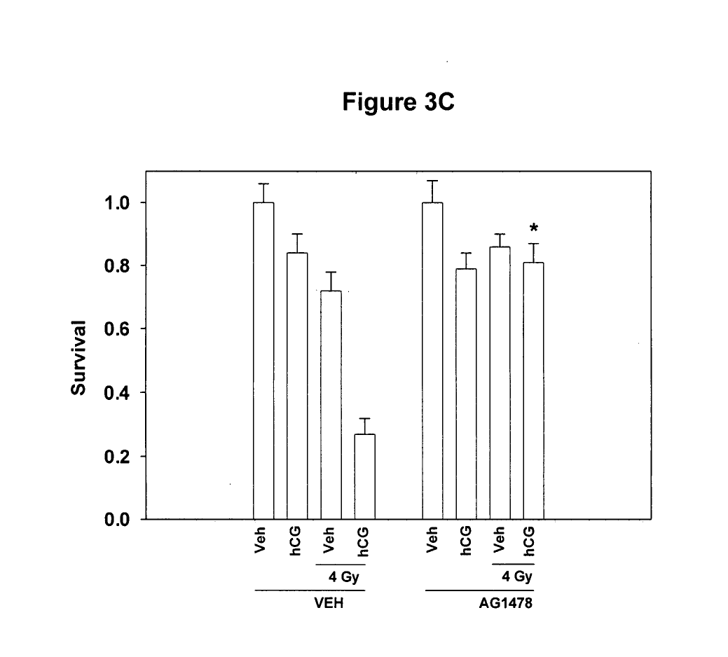

PARP1 Activation and Expression Following Treatment with a PARP Inhibitor (PJ-34), hCG and Radiation. LNCaP cells were cultured as described above. Twenty four hours after plating, the cells were pre-treated with vehicle (VEH, DMSO) or with the PARP inhibitor PJ-34 (5 μM) followed 30 minutes later by treatment with vehicle (VEH, PBS) or with hCG (2 mU/ml). Thirty minutes after hCG treatment, cells were irradiated (4 Gy). Cells were isolated 96 hours after irradiation by trypsinization, the attached and floating cells combined, and cell viability determine by trypan blue exclusion assays using a light microscope. Cells were then lysed in SDS PAGE sample buffer and equal protein concentrations subjected to SDS PAGE followed by immunoblotting to determine the PARP1 ADP-ribosylation status (activity status) in the presence or absence of PJ-34. Results indicated that inhibition of PARP function using low concentrations of a highly specific inhibitor of PARP enzymes, PJ-34, or using siRNA to knock down PARP1 expression, abolished the potentiation of hCG lethality by radiation (FIG. 3A).

PARP1 and AIF Expression Following Treatment with a PARP Inhibitor (PJ-34) and hCG. LNCaP cells were cultured as described in Example 1. Twelve hours after plating the cells were transfected using Lipofectamine with either a scrambled siRNA (SCR) or with siRNA molecules to suppress PARP1 or AIF expression (siPARP; siAIF; 10 nM each). Twenty four hours after transfection, as indicated, cells were treated with hCG (2 mU/ml) or vehicle (VEH, PBS)), and 30 min later irradiated (4 Gy). Cells were isolated 96 hours after irradiation by trypsinization, the attached and floating cells combined, and cell viability determine by trypan blue exclusion assays using a light microscope. Cells were then lysed in SDS PAGE sample buffer and equal protein concentrations subjected to SDS PAGE followed by immunoblotting to determine PARP1 and AIF expression in the presence or absence of siPARP and siAIF, siRNA molecules, respectively. PARP-induced cell death has been linked by some groups to the cytotoxic actions of AIF. Results indicated that suppression of AIF expression reduced the lethality of combined hCG and radiation exposure by ˜50% (FIG. 3B) (Kolthur-Seetharam et al., Cell Cycle, 5:873-877, 2006).

Apoptosis Assay. LNCaP cells were cultured as described above. Twenty four hours after plating (250-2,500 cells/well of a 6 well plate), cells were pre-treated with vehicle (VEH, DMSO) or a small molecule inhibitor, AG1478 (1 μM) and thirty minutes later treated with hCG (2 mU/ml). Thirty minutes after hCG exposure, cells were irradiated (4 Gy). Ninety six hours after irradiation, media containing hCG and AG1478 was removed and fresh media lacking hCG/AG1478 added. Colonies were permitted to form over the following 28 days after which the media was removed, the cells fixed and stained with crystal violet. Groups of >50 cells were considered to be colonies. Results indicated that inhibition of ERBB1 function by use of AG1478 (removed 96 h after hCG and radiation exposure) also prevented the greater than additive induction of cell killing caused by combined hCG and radiation exposure (FIG. 3C).

Results. Collectively, these findings indicate that hCG and radiation exposure interact in an ERBB1-dependent manner to promote high levels of PARP activation which were responsible for LNCaP cell radiosensitization by hCG.

### EXAMPLE 5

### Inhibition of PI3K Enhances the Toxicity of hCH in LNCaP Cells

To further investigate the role of signal transduction pathways in the survival response of hCG treated prostate cancer cells, the abilities of ERBB1, MEK½ and PI3K inhibitors to modify cell viability in hCG treated LNCaP cells were examined.

Cell culture. LNCaP cells were purchased from the ATCC were cultured at 37° C. (5% (v/v CO₂) in vitro using RPMI supplemented with 10% (v/v) fetal calf serum.

SDS-PAGE and Immunoblotting. The cells were plated at 5×10⁵ cells/cm² and treated with drugs at the indicated concentrations and after the indicated time of treatment, lysed in whole-cell lysis buffer (0.5 M Tris-HCl, pH 6.8, 2% SDS, 10% glycerol, 1% β-mercaptoethanol, 0.02% bromophenol blue), and the samples were boiled for 30 minutes. The boiled samples were loaded onto 10-14% SDS-PAGE and electrophoresis was run overnight. Proteins were electrophoretically transferred onto 0.22 μm nitrocellulose, and immunoblotted with various primary antibodies against different proteins. All immunoblots were visualized by enhanced chemiluminescence (ECL).

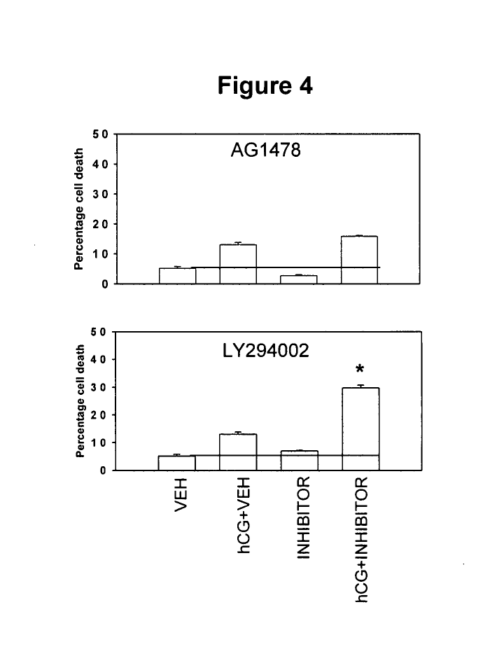

Apoptosis assay. LNCaP cells were cultured as described above. Twenty four hours after plating, cells were pre-treated with vehicle (VEH, DMSO) or with either the PI3K inhibitor LY294002 (10 μM) or the small molecule inhibitor, AG1478 (1 μM), followed 30 minutes later by treatment with vehicle (VEH, PBS) or with hCG (2 mU/ml). Cells were isolated by trypsinization 96 hours after irradiation, the attached and floating cells combined, and cell viability determine by trypan blue exclusion assays using a light microscope. Results indicated that treatment of cells with the ERBB1 inhibitor AG1478 did not significantly alter cell viability following hCG exposure indicating that hCG induced activation of ERBB1, per se, is not a toxic signal (FIG. 4). Thus, inhibition of PI3K signaling using a small molecule inhibitor enhanced hCG lethality in LNCaP cells in a greater than additive fashion.

### EXAMPLE 6

### The HMG CoA Reductase Inhibitor Lovastatin Enhances the Toxicity of hCG in a Greater than Additive Fashion in Multiple Carcinoma Cell Types

This example demonstrates that lovastatin enhances the toxicity of hCG.

Cell culture. LNCaP cells, 22RW1 (prostate cancer) and SKOV3 (ovarian carcinoma) cells were purchased from the ATCC were cultured at 37° C. (5% (v/v CO₂) in vitro using RPMI supplemented with 10% (v/v) fetal calf serum.

SDS-PAGE and Immunoblotting. The cells were plated at 5×10⁵ cells/cm² and treated with drugs at the indicated concentrations and after the indicated time of treatment, lysed in whole-cell lysis buffer (0.5 M Tris-HCl, pH 6.8, 2% SDS, 10% glycerol, 1% β-mercaptoethanol, 0.02% bromophenol blue), and the samples were boiled for 30 minutes. The boiled samples were loaded onto 10-14% SDS-PAGE and electrophoresis was run overnight. Proteins were electrophoretically transferred onto 0.22 μm nitrocellulose, and immunoblotted with various primary antibodies against different proteins. All immunoblots were visualized by enhanced chemiluminescence (ECL).

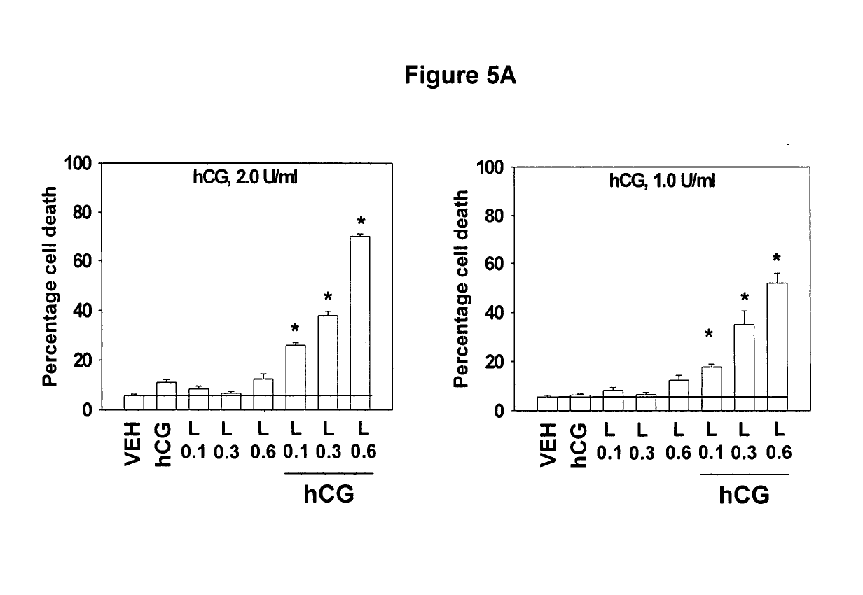

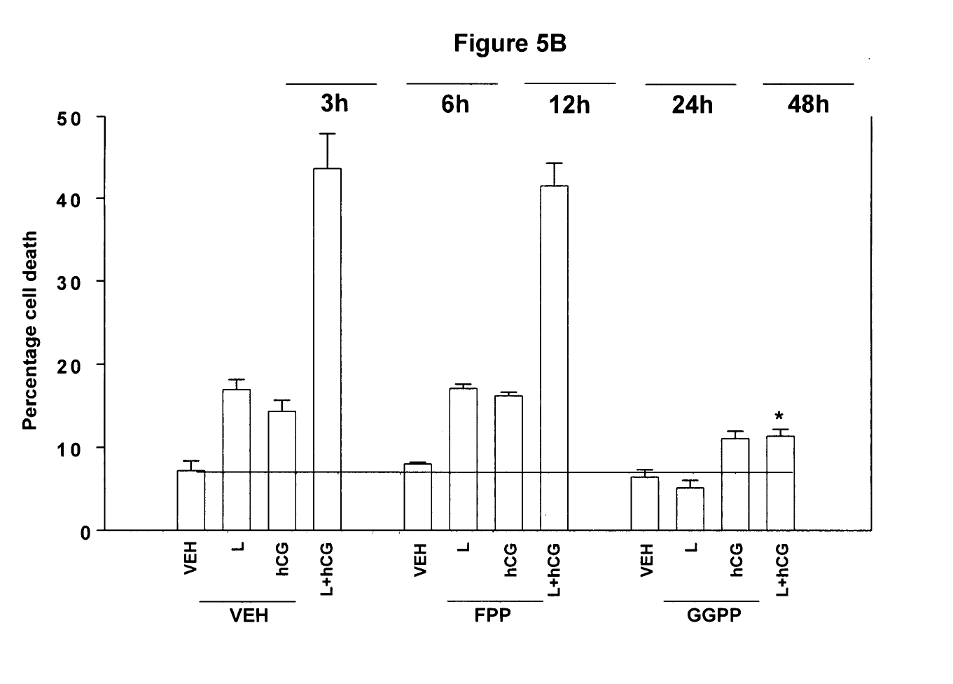

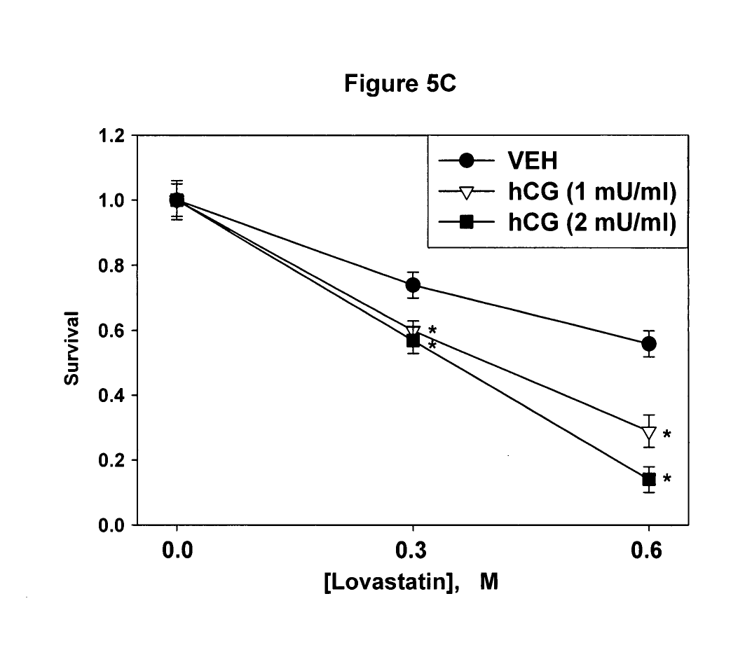

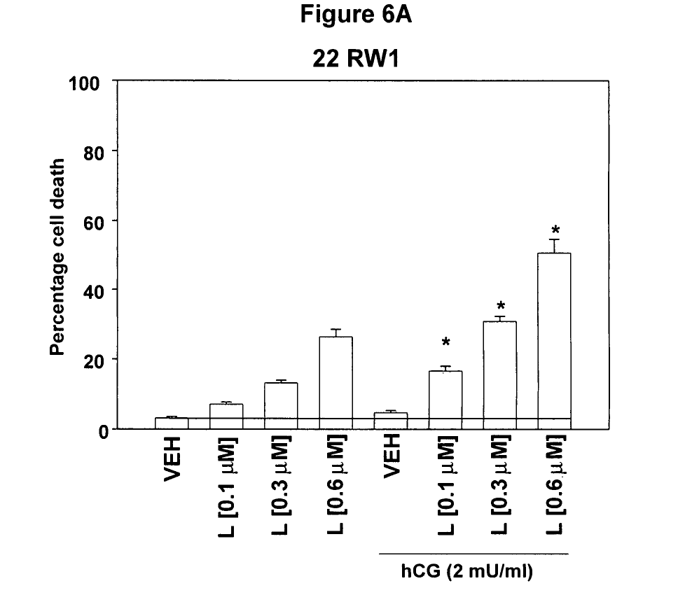

Activation of ERK ½ and AKT and the Expression of ERK2 Following Treatment with hCG and Lovastatin. LNCaP cells were cultured as described above. Twenty four hours after plating, cells were pre-treated with vehicle (VEH, DMSO) or with the HMG CoA reductase inhibitor lovastatin (L; 0, 0.1, 0.3, 0.6 μM), followed 30 minutes later by treatment with vehicle (VEH, PBS) or with hCG (1 or 2 mU/ml)). Cells were isolated 96 hours after irradiation by trypsinization, the attached and floating cells combined, and cell viability determine by trypan blue exclusion assays using a light microscope. Forty eight hours after hCG and lovastatin (L 0-0.6 μM) exposure, the cells were lysed in SDS PAGE sample buffer and equal protein concentrations subjected to SDS PAGE followed by immunoblotting to determine the phosphorylation status (activity status) of ERK½ and AKT (S473) and the expression level of ERK2 protein. Results indicated that treatment of LNCaP cells with low clinically achievable concentrations of lovastatin (0.1-0.6 μM) enhanced the lethality of hCG to an extent greater than was observed using a PI3K inhibitor (FIG. 5A).

Activation of ERK ½ and AKT and the Expression of ERK2 Following Treatment with hCG and Lovastatin. PC3 prostate carcinoma cells were cultured as described above. Twenty four hours after plating, cells were pre-treated with vehicle (VEH, DMSO) or with lovastatin (L; 0, 0.1-1.0 μM), followed 30 min later by treatment with vehicle (PBS) or with hCG (1 mU/ml). Cells were isolated 96 hours after hCG treatment by trypsinization, the attached and floating cells combined, and cell viability determine by trypan blue exclusion assays using a light microscope. Forty eight hours after hCG and lovastatin (L 0-1.0 μM) exposure, the cells were lysed in SDS PAGE sample buffer and equal protein concentrations subjected to SDS PAGE followed by immunoblotting to determine the phosphorylation status (activity status) of ERK½ and AKT (S473) and the expression level of ERK2 protein.

Activation of RhoA and the Expression of ERK2 Following Treatment with hCG and Lovastatin. LNCaP cells were cultured as described above. Twenty three hours after plating, cells were treated with either the control, vehicle (VEH, DMSO), or farnesyl pyrophosphate (FPP; 1 μM) or geranylgeranyl pyrophosphate (GGPP, 1 μM). Twenty four hours after plating, cells were pre-treated with vehicle (VEH, DMSO) or with lovastatin (L; 0.6 μM), followed 30 min later by treatment with vehicle (PBS) or with hCG (2 mU/ml). Cells were isolated 96 hours after hCG treatment by trypsinization, the attached and floating cells combined, and cell viability determine by trypan blue exclusion assays using a light microscope. Forty eight hours after hCG and lovastatin (L 0.6 μM) exposure, the cells were lysed in SDS PAGE sample buffer and equal protein concentrations subjected to SDS PAGE followed by immunoblotting to determine the lipid modification status (activity status) of Rho A and the expression level of ERK2 protein. Results indicated that incubation of cells with geranylgeranyl pyrophosphate (GGPP), but not farnesyl pyrophosphate (FPP), abolished the lethal interaction between hCG and lovastatin, indicating that the functional inhibition of a geranylgernaylated protein by lovastatin enhanced hCG lethality (FIG. 5B) (van de Donk et al., Blood 102: 3354-3362 (2003); Fritz et al., Curr Cancer Drug Targets 6: 1-14 (2006); Holstein et al., Cancer Chemother Pharmacol; 57: 155-164 (2006); and Thibault et al., Clin Cancer Res 2: 483-490 (1996)). Identical data LNCaP cell killing data for hCG and lovastatin exposure were obtained when a geranylgernayltransferase inhibitor (GGTI-286, 10 μM) was used in place of lovastatin. While not intending to be bound by any particular theory of invention, this result possibly implies that the statin causes its effect by reducing the geranylgeranylation of a protein in an LNCaP cell, rather than the farnesylation of a protein. In some cells the statin may enhance the effects of the chorionic gonadotropin by blocking farnesylation in which case the use of a farnesyl transferase inhibitor will be of further use.

Bioassay for cell survival. LNCaP cells were cultured as described above. Twenty four hours after plating (250-2,500 cells/well of a 6 well plate), cells were treated with lovastatin (L, 0-0.6 μM) 30 min prior to hCG (2 mU/ml). Ninety six hours after hCG and lovastatin treatment, media containing hCG and lovastatin was removed and fresh media lacking hCG and lovastatin was added. Colonies were permitted to form over the following 28 days after which the media was removed, the cells fixed and stained with crystal violet. Groups of >50 cells were considered to be colonies. Results indicated that LNCaP cell death 96 hours after exposure were re-capitulated in colony formation assays assessing cell growth 21-28 days after lovastatin and hCG treatment (FIG. 5C).

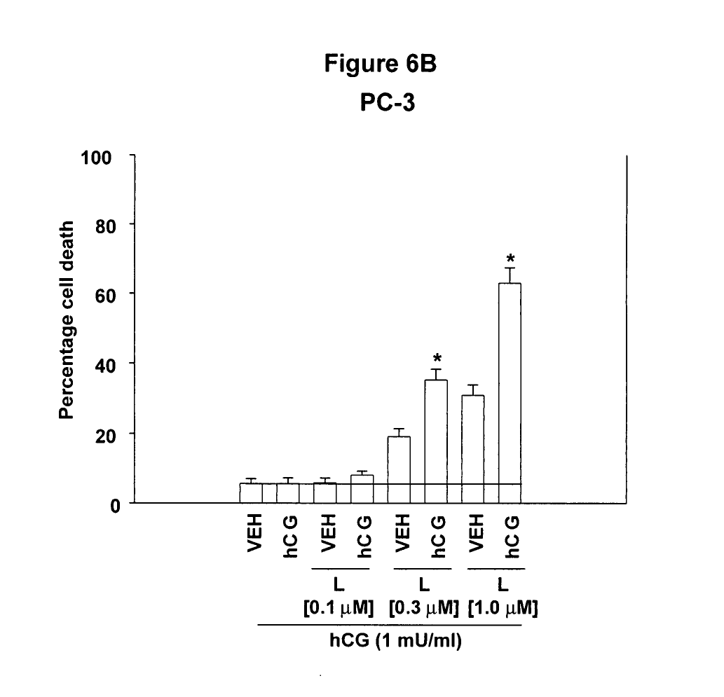

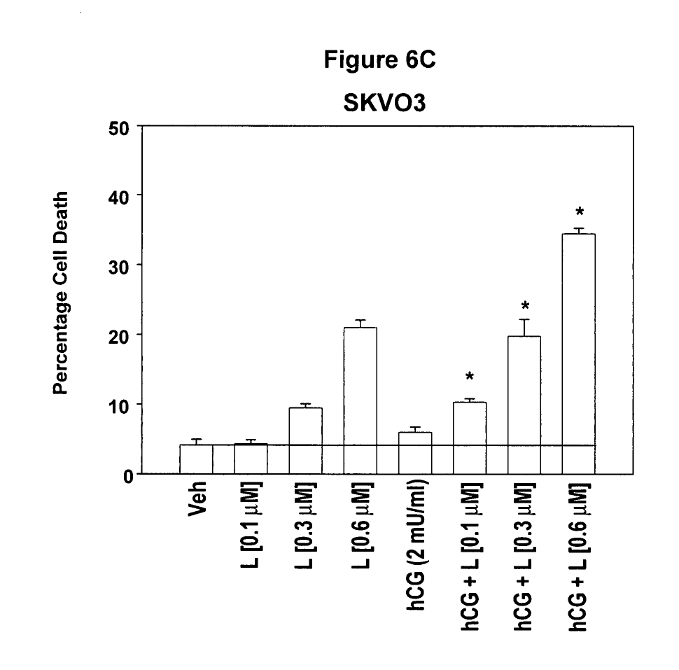

Apoptosis assay in PC3, 22RW1 and SKOV3 cells. PC3, 22RW1 and SKOV3 cells were cultured as described above. Twenty four hours after plating (250-2,500 cells/well of a 6 well plate), cells were pre-treated with vehicle (VEH, DMSO) or with lovastatin (L; 0, 0.1, 0.3, 0.6 μM), followed 30 min later by treatment with vehicle (VEH, PBS) or with hCG (1-2 mU/ml). Cells were isolated 96 hours after hCG treatment by trypsinization, the attached and floating cells combined, and cell viability determined by trypan blue exclusion assays using a light microscope. Similar cell killing data to those in LNCaP cells was obtained by combining hCG and lovastatin in 22RW1 and PC-3 prostate cancer cells as well as in SKOV3 ovarian carcinoma cells (FIGS. 6A-6C).

Results. Collectively, these findings demonstrate that low clinically relevant concentrations of lovastatin enhance the lethality of hCG in multiple androgen-dependent and -independent carcinoma cell lines in vitro which occurs by suppression of protein geranylgeranylation.

### EXAMPLE 7

### Lovastatin and hCG Treatment Causes Inactivation of AKT and JNK½-Dependent Cell Killing

This example demonstrates the impact of lovastatin and hCG on the activation status of signal transduction pathways in LNCaP cells.

Cell culture. LNCaP cells were purchased from the ATCC were cultured at 37° C. (5% (v/v CO₂) in vitro using RPMI supplemented with 10% (v/v) fetal calf serum.

Recombinant adeno viral vectors, generation and infection in vitro. Recombinant adenoviruses were generated to express constitutively activated and dominant negative AKT and MEK1 proteins, dominant negative ERBB1 (COOH-terminal 533 amino acid deletion; CD533), dominant negative JNK1 (Hagan et al., Clin Cancer Res. 10: 5724-5731, 2004; Caron et al., Mol Cancer Ther. 4: 257-270, 2005; Caron et al., Mol Cancer Ther. 4: 243-255, 2005; and Yacoub et al., Mol Pharmacol 70: 589-603, 2006). LCNaP cells were infected with these adenoviruses at an approximate multiplicity of infection (m.o.i.) of 25. Cells were further incubated for 24 hours to ensure adequate expression of transduced gene products prior to any drug exposures/assays.

SDS-PAGE and Immunoblotting. The cells were plated at 5×10⁵ cells/cm² and treated with drugs at the indicated concentrations and after the indicated time of treatment, lysed in whole-cell lysis buffer (0.5 M Tris-HCl, pH 6.8, 2% SDS, 10% glycerol, 1% β-mercaptoethanol, 0.02% bromophenol blue), and the samples were boiled for 30 minutes. The boiled samples were loaded onto 10-14% SDS-PAGE and electrophoresis was run overnight. Proteins were electrophoretically transferred onto 0.22 μm nitrocellulose, and immunoblotted with various primary antibodies against different proteins. All immunoblots were visualized by enhanced chemiluminescence (ECL).

Activation of ERK ½, JNK ½ and AKT and the Expression of ERK 2 Following Treatment with hCG and Lovastatin. LNCaP cells were cultured as described above. Twenty four hours after plating, cells were pre-treated with vehicle (VEH, DMSO) or with lovastatin (L; 0.6 μM), followed 30 minutes later by treatment with vehicle (VEH, PBS) or with hCG (2 mU/ml). At the indicated times after treatment (12-96 h), the cells were isolated, lysed in SDS PAGE sample buffer and equal protein concentrations subjected to SDS PAGE followed by immunoblotting to determine the phosphorylation status (activity status) of ERK½, JNK½, AKT (S473) and the expression level of ERK2 protein. Results indicated that lovastatin treatment within 48 hours had suppressed basal AKT activity, suppressed hCG induced ERK½ and AKT activation, and promoted JNK½ activation following exposure combined exposure to the agents.

Expression of I.B., XIAP, BCL-XL and ERK2 Following Treatment with hCG and Lovastatin. LNCaP cells were cultured as described above. Twenty four hours after plating, cells were pretreated with vehicle (VEH, DMSO) or with lovastatin (L; 0.6 μM), followed 30 minutes later by treatment with vehicle (VEH, PBS) or with hCG (2 mU/ml). At 48 hours or 96 hours after treatment, the cells were isolated, lysed in SDS PAGE sample buffer and equal protein concentrations subjected to SDS PAGE followed by immunoblotting to determine the expression of I.B, XIAP and BCL-XL, and the total expression of ERK2 protein.

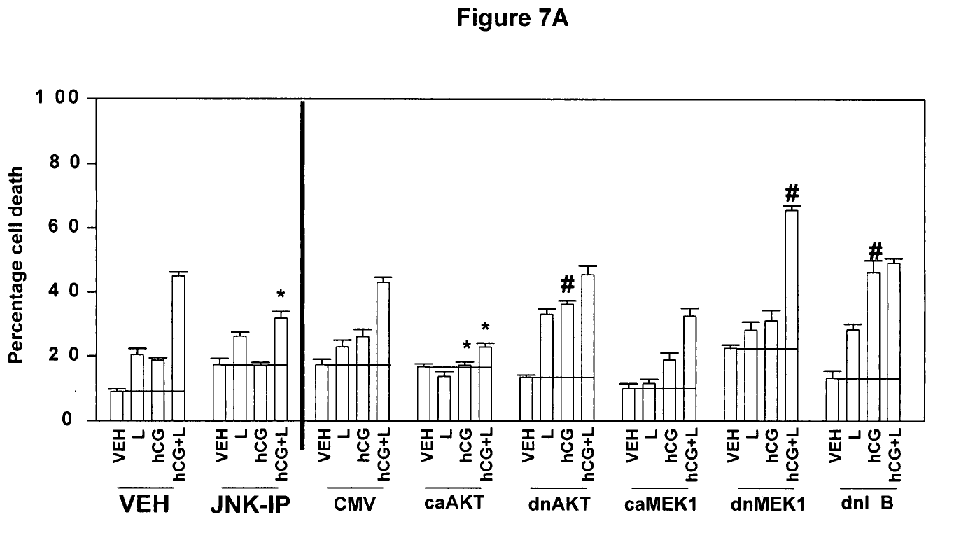

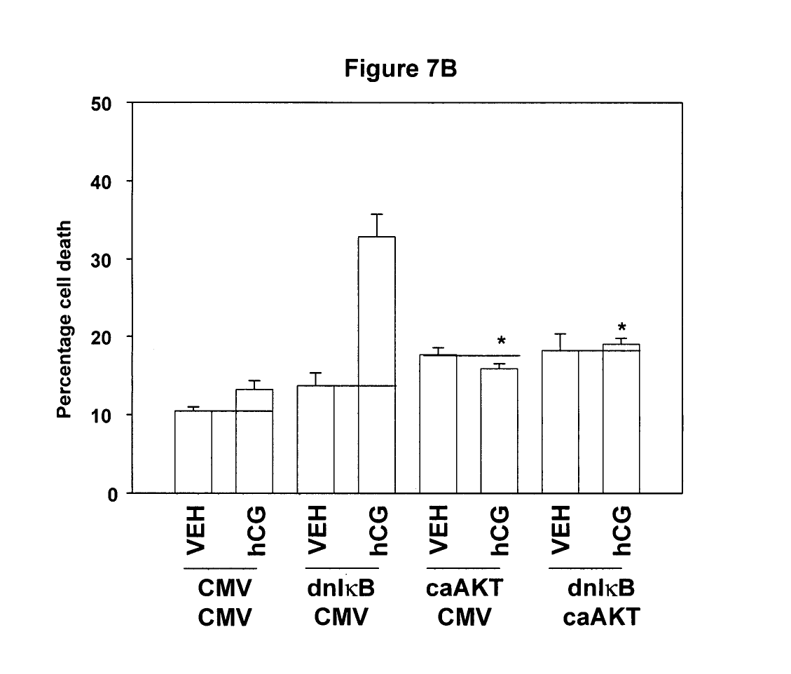

Apoptosis assay in LNCaP Cells. (1) LNCaP cells were cultured as described above. Twelve hours after plating, cells were infected with either empty vector virus (CMV) or with viruses to express dominant negative AKT, dominant negative MEK1, dominant negative IkB, constitutively active AKT, and constitutively active MEK1 at a multiplicity of infection (m.o.i.) of 25. In other parallel plates of cells, 24 h after plating, cells were pretreated 30 minutes prior to any additional manipulation with either vehicle (VEH, DMSO) or a JNK½ inhibitory peptide (JNK-IP, 10 μM). All cells were then treated with vehicle (VEH, DMSO) or with lovastatin (L; 0.6 μM), followed 30 minutes later by treatment with vehicle (VEH, PBS) or with hCG (2 mU/ml). Cells were isolated 96 hours after hCG treatment by trypsinization, the attached and floating cells combined, and cell viability determine by trypan blue exclusion assays using a light microscope. Results indicated that incubation of cells with a cell permeable fragment of the JNK inhibitory protein significantly suppressed hCG lethality as a single agent and hCG and lovastatin lethality in combination. Expression of constitutively active AKT significantly suppressed cell killing by hCG and by hCG and lovastatin treatment. Expression of dominant negative AKT or dominant negative IkB enhanced the lethality of lovaststain or hCG as single agents and abolished their toxic interaction. See FIG. 7A.

(2) LNCaP cells were cultured as described above. Twelve hours after plating, cells were infected with either empty vector virus (CMV) or with viruses to express dominant negative IkB and/or constitutively active AKT each at a multiplicity of infection (m.o.i.) of 25. The total m.o.i. for virus infection was 50. All cells were then treated with vehicle (VEH, DMSO) or with lovastatin (L; 0.6 μM), followed 30 minutes later by treatment with vehicle (VEH, PBS) or with hCG (2 mU/ml). Cells were isolated 96 hours after hCG treatment by trypsinization, the attached and floating cells combined, and cell viability determine by trypan blue exclusion assays using a light microscope. Results indicated that treatment with lovastatin and hCG increased the expression of I.B, and decreased expression of proteins whose expression is known to be regulated by NF.B; the caspase inhibitor XIAP and the mitochondrial protective protein BCL-XL. Expression of constitutively active AKT protected LNCaP cells from hCG toxicity in the absence and in the presence of co-expressed dominant negative I.B, suggesting that AKT and NF.B are independent/over-lapping survival pathways against hCG toxicity and that activation of AKT can compensate for the loss of NF.B function (FIG. 7B).

### EXAMPLE 8

### The Effects of Lovastatin on hCG Lethality

This example investigates the mechanism by which lovastatin potentiates hCG lethality.

Cell culture. LNCaP cells were purchased from the ATCC were cultured at 37° C. (5% (v/v CO₂) in vitro using RPMI supplemented with 10% (v/v) fetal calf serum.

Recombinant adeno viral vectors, generation and infection in vitro. Recombinant adenoviruses were generated to express constitutively activated and dominant negative AKT and MEK1 proteins, dominant negative ERBB1 (COOH-terminal 533 amino acid deletion; CD533), dominant negative JNK1 (Hagan et al., Clin Cancer Res. 10: 5724-5731, 2004; Caron et al., Mol Cancer Ther. 4: 257-270, 2005; Caron et al., Mol Cancer Ther. 4: 243-255, 2005; and Yacoub et al., Mol Pharmacol 70: 589-603, 2006). LCNaP cells were infected with these adenoviruses at an approximate multiplicity of infection (m.o.i.) of 25. Cells were further incubated for 24 hours to ensure adequate expression of transduced gene products prior to any drug exposures/assays.

SDS-PAGE and Immunoblotting. The cells were plated at 5×10⁵ cells/cm² and treated with drugs at the indicated concentrations and after the indicated time of treatment, lysed in whole-cell lysis buffer (0.5 M Tris-HCl, pH 6.8, 2% SDS, 10% glycerol, 1% β-mercaptoethanol, 0.02% bromophenol blue), and the samples were boiled for 30 minutes. The boiled samples were loaded onto 10-14% SDS-PAGE and electrophoresis was run overnight. Proteins were electrophoretically transferred onto 0.22 μm nitrocellulose, and immunoblotted with various primary antibodies against different proteins. All immunoblots were visualized by enhanced chemiluminescence (ECL).

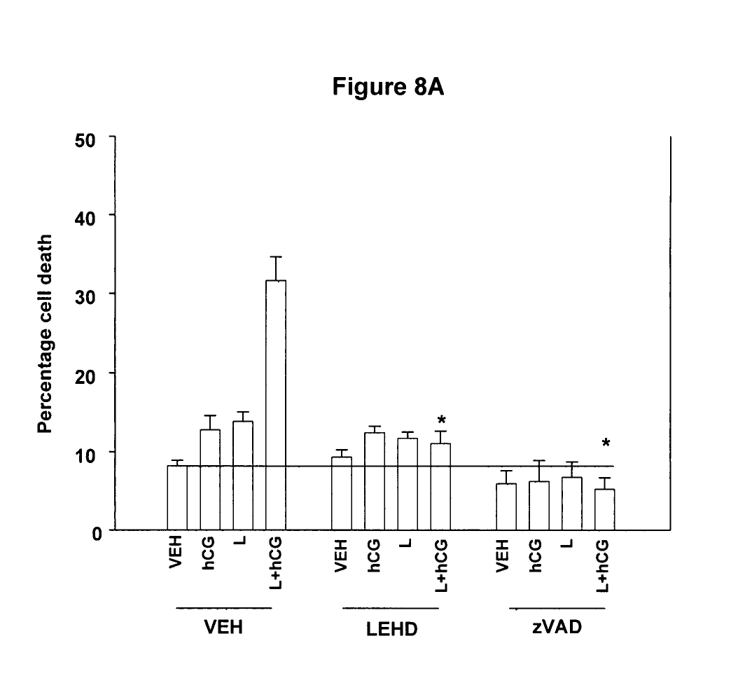

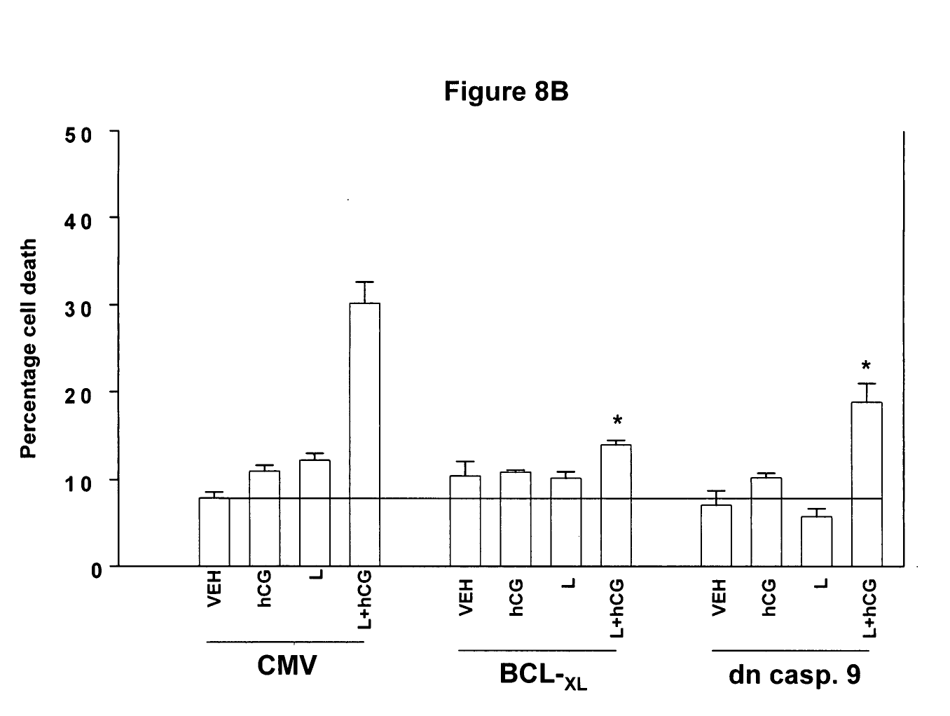

Apoptosis assay. LNCaP cells were cultured as described above. Twenty three hours after plating, the cells were treated with either vehicle (VEH, DMSO), with the caspase 9 inhibitor LEHD (50 μM) or the pan-caspase inhibitor zVAD (50 μM). Twenty four hours after plating, cells were pre-treated with vehicle (VEH, DMSO) or with lovastatin (L; 0.6 μM), followed 30 minutes later by treatment with vehicle VEH, PBS) or with hCG (2 mU/ml). Cells were re-supplemented with caspase inhibitors every 24 hours. The cells were isolated 96 hours after hCG treatment by trypsinization, the attached and floating cells combined, and cell viability determine by trypan blue exclusion assays using a light microscope. Results indicated that inhibition of caspase 8 function did not alter the survival of cells treated with hCG, lovastatin or the combination of hCG and lovastatin. Treatment of cells with either the pan-caspase inhibitor zVAD or the caspase 9 inhibitor LEHD reduced the lethality of the lovastatin and hCG combination (FIG. 8A).

Expression of ERK2 Following Treatment with hCG and Lovastatin. LNCaP cells were cultured as described above. Twelve hours after plating, the cells were infected with either empty vector virus (CMV) or with a virus to express constitutively active AKT at a multiplicity of infection (m.o.i.) of 25. Cells were then treated with vehicle (VEH, DMSO) or with lovastatin (L; 0.6 μM), followed 30 minutes later by treatment with vehicle (VEH, PBS) or with hCG (2 mU/ml). Cells were isolated 48 hours after hCG treatment by lysis in SDS PAGE sample buffer and equal protein concentrations subjected to SDS PAGE followed by immunoblotting to determine the phosphorylation status (activity status) of JNK½ and AKT (S473) and the expression level of ERK2 protein. Results indicated that expression of constitutively active AKT suppressed JNK½ activity and inhibition of JNK½ signaling suppressed release of cytochrome c into the cytosol.

LNCaP cells were cultured as described above. Twelve hours after plating, cells were infected with either empty vector virus (CMV) or with viruses to express dominant negative caspase 9 or over-express BCL-XL at a multiplicity of infection (m.o.i.) of 25. Twenty four hours after plating, cells were then treated with vehicle (VEH, DMSO) or with lovastatin (L; 0.6 μM), followed 30 minutes later by treatment with vehicle (VEH, PBS) or with hCG (2 mU/ml). Cells were isolated 96 hours after hCG treatment by trypsinization, the attached and floating cells combined, and cell viability determine by trypan blue exclusion assays using a light microscope. Results indicated that over-expression of dominant negative caspase 9 reduced the potentiation of hCG lethality by lovastatin. Combined exposure of LNCaP cells to hCG and lovastatin caused lower expression levels of the anti-apoptotic protein BCL-XL, and over-expression of BCL-XL maintained LNCaP survival when cells were treated with hCG and lovastatin (FIG. 8B).

LNCaP cells were cultured as described above. Twelve hours after plating, cells were infected with either empty vector virus (CMV) or with viruses to express dominant negative IkB and/or constitutively active AKT each at a multiplicity of infection (m.o.i.) of 25, as indicated. The total m.o.i. for virus infection was 50. All cells were then treated with vehicle (VEH, DMSO) or with lovastatin (L; 0.6 μM), followed 30 minutes later by treatment with vehicle (PBS) or with hCG (2 mU/ml). Cells were isolated 48 hours after hCG treatment lysed in SDS PAGE sample buffer and equal protein concentrations subjected to SDS PAGE followed by immunoblotting to determine the expression of I.B and BCL-, the phosphorylation of AKT (S473) and the total expression of ERK2 protein. Results indicated that expression of dominant negative IkB suppressed BCL-XL levels, that were rescued by expression of constitutively active AKT, suggesting that hCG and lovastatin lethality is in part mediated by inhibition of AKT and of NF.B signaling, leading to reduced BCL-XL expression and increased mitochondrial dysfunction (FIG. 8B) (Mayo et al., J Biol Chem. 278: 18980-18989 (2003)).

### DISCUSSION

As discussed herein, lovastatin was used at concentrations of 0.1-0.6 μM, which are achievable in human plasma (Holstein et al., 57: 155-164 (2006) and Thibault et al., Clin Cancer Res 2: 483-490 (1996)). At these concentrations, the lipid modification of Rho A i.e. geranylgeranylation, was suppressed by lovastatin in LNCaP cells in vitro. In LNCaP cells, low concentrations of lovastatin reduced AKT activity and in combination with hCG, promoted activation of JNK½ and increased expression of I.B. The balance in cell signaling between pro-survival and pro-death signaling pathways has been argued by many groups using a wide variety of agents to cause cell death (reviewed in Dent et al., Oncogene 22: 5885-5896 (2003) and Dent et al., Radiat Res. 59: 283-300 (2003)). Based on prior studies by others (e.g. van de Donk et al., Blood 102: 3354-3362 (2003); Li et al., Front Biosci. 10: 236-243 (2005); Mayo et al., J Biol Chem. 278: 18980-18989 (2003) and Wu et al., Cancer Res 64: 6461-6468 (2004)), it was initially hypothesized that lovastatin-induced inhibition of AKT function and increased expression of I.B were linked in a simple linear pathway to the observed reduction in BCL-XL and XIAP levels. i.e. reduced AKT activity suppressed NF.B activity and expression of dominant negative I.B reduced expression of BCL-XL XL levels. These findings argue that in LNCaP cells, AKT signaling represents a primary negative regulator of lovastatin and hCG lethality, and that modulation of NF.B function is secondary to cell survival processes. It is likely that in other prostate cancer cell types, where NF.B is more active, that NF.B signaling may play a more important role in maintaining viability after lovaststin and hCG exposure.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed is:

1. A method of treating cancer in a subject comprising administering to said subject a therapeutically effective amount of a composition comprising a chorionic gonadotropin or a therapeutically active fragment or analogue thereof in combination with an HMG CoA reductase inhibitor.

2. The method of claim 1, wherein the chorionic gonadotropin is hCG or a therapeutically active fragment or analogue thereof.

3. The method of claim 1, wherein the therapeutically active fragment is a beta subunit of hCG.

4. The method of claim 1, wherein the HMG CoA reductase inhibitor is a statin.

5. The method of claim 1, wherein the HMG CoA reductase inhibitor is selected from the group consisting of lovastatin, simvastatin, pravastatin, fluvastatin, atorvastatin, itavastatin, rosuvastatin and rivastatin.

6. The method of claim 1 wherein the HMG CoA reductase inhibitor is lovastatin.

7. The method of claim 1, wherein the cancer is selected from the group consisting of, myxoid and round cell carcinoma, Ewing's sarcoma, cancer metastases, including lymphatic metastases, squamous cell carcinoma, esophageal squamous cell carcinoma, oral carcinoma, multiple myeloma, lymphocytic leukemia, acute nonlymphocytic leukemia, chronic lymphocytic leukemia, chronic myelocytic leukemia, and hairy cell leukemia, effusion lymphomas, thymic lymphoma lung cancer, small cell carcinoma of the lungs, cutaneous T cell lymphoma, Hodgkin's lymphoma, non Hodgkin's lymphoma, cancer of the adrenal cortex, non-small cell lung cancers, breast cancer, stomach cancer, colon cancer, colorectal cancer, colorectal neoplasia, pancreatic cancer, liver cancer, bladder cancer, primary superficial bladder tumors, invasive transitional cell carcinoma of the bladder, muscle invasive bladder cancer, prostate cancer, ovarian carcinoma, primary peritoneal epithelial neoplasms, cervical carcinoma, uterine endometrial cancers, vaginal cancer, cancer of the vulva, uterine cancer, solid tumors in the ovarian follicle, testicular cancer, penile cancer, kidney cancer, renal cell carcinoma, brain cancer, neuroblastoma, astrocytic brain tumors, gliomas, metastatic tumor cell invasion in the central nervous system, osteomas, osteosarcomas, malignant melanoma, tumor progression of human skin keratinocytes, basal cell carcinoma, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

8. The method of claim 1, wherein the cancer is prostate cancer.

9. The method of claim 1, further comprising the step of treating said cancer with radiation after administration of the chorionic gonadotropin and HMG CoA reductase inhibitor composition.

10. The method of claim 9 wherein the radiation is external beam X-ray radiation.

11. The method of claim 9 wherein the radiation is administered by means of brachytherapy.

12. The method of claim 1, wherein the composition further comprises an additional agent that inhibits NF-kappa-B activity.

13. The method of claim 1, wherein the composition further comprises an additional agent that inhibits PI-3-kinase activity.

14. The method of claim 1, wherein the composition further comprises an additional agent that inhibits AKT activity.

15. The method of claim 1, wherein the composition further comprises an additional agent which is a geranylgeranyltransferase inhibitor (GGTI).

16. The method of claim 1, wherein the composition further comprises an additional agent which is a farnesyl transferase inhibitor (FTI).

17. A pharmaceutical composition for the treatment of cancer comprising a therapeutically effective amount of a composition comprising a chorionic gonadotropin or a therapeutically active fragment or analogue thereof in combination with an HMG CoA reductase inhibitor.

18. The composition of claim 17, wherein the chorionic gonadotropin is hCG or a therapeutically active fragment or analogue thereof.

19. The composition of claim 17, wherein the therapeutically active fragment is a beta subunit of hCG.

20. The composition of claim 17, wherein the HMG CoA reductase inhibitor is a statin.

21. The composition of claim 20, wherein the HMG CoA reductase inhibitor is selected from the group consisting of lovastatin, simvastatin, pravastatin, fluvastatin, atorvastatin, itavastatin, rosuvastatin and rivastatin.

22. The composition of claim 21, wherein the statin is lovastatin.

23. The composition of claim 17, which further comprises an additional agent that inhibits NF-kappa-B activity.

24. The composition of claim 17, which further comprises an additional agent that inhibits PI-3-kinase activity.

25. The composition of claim 17, which further comprises an additional agent that inhibits AKT activity.

26. The composition of claim 17, which further comprises an additional agent which is a geranylgeranyltransferase inhibitor (GGTI).

27. The composition of claim 17, which further comprises an additional agent which is a farnesyltransferase inhibitor (FTI).

28. A method of treating cancer in a subject comprising administering to said subject a therapeutically effective amount of a composition comprising a chorionic gonadotropin or a therapeutically active fragment or analogue thereof in combination with a geranylgeranyltransferase inhibitor.

29. The method of claim 28, wherein the chorionic gonadotropin is hCG or a therapeutically active fragment or analogue thereof.

30. The method of claim 28 wherein the therapeutically active fragment is a beta subunit of hCG.

31. The method of claim 28, wherein the cancer is selected from the group consisting of, myxoid and round cell carcinoma, Ewing's sarcoma, cancer metastases, including lymphatic metastases, squamous cell carcinoma, esophageal squamous cell carcinoma, oral carcinoma, multiple myeloma, lymphocytic leukemia, acute nonlymphocytic leukemia, chronic lymphocytic leukemia, chronic myelocytic leukemia, and hairy cell leukemia, effusion lymphomas, thymic lymphoma lung cancer, small cell carcinoma of the lungs, cutaneous T cell lymphoma, Hodgkin's lymphoma, non Hodgkin's lymphoma, cancer of the adrenal cortex, non-small cell lung cancers, breast cancer, stomach cancer, colon cancer, colorectal cancer, colorectal neoplasia, pancreatic cancer, liver cancer, bladder cancer, primary superficial bladder tumors, invasive transitional cell carcinoma of the bladder, muscle invasive bladder cancer, prostate cancer, ovarian carcinoma, primary peritoneal epithelial neoplasms, cervical carcinoma, uterine endometrial cancers, vaginal cancer, cancer of the vulva, uterine cancer, solid tumors in the ovarian follicle, testicular cancer, penile cancer, kidney cancer, renal cell carcinoma, brain cancer, neuroblastoma, astrocytic brain tumors, gliomas, metastatic tumor cell invasion in the central nervous system, osteomas, osteosarcomas, malignant melanoma, tumor progression of human skin keratinocytes, basal cell carcinoma, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

32. The method of claim 28, wherein the cancer is prostate cancer.

33. The method of claim 28, further comprising the step of treating said cancer with radiation after administration of the chorionic gonadotropin and geranylgeranyltransferase inhibitor composition.

34. The method of claim 33, wherein the radiation is external beam X-ray radiation.

35. The method of claim 33, wherein the radiation is administered by means of brachytherapy.

36. The method of claim 28, wherein the composition further comprises an additional agent that inhibits NF-kappa-B activity.

37. The method of claim 28, wherein the composition further comprises an additional agent that inhibits PI-3-kinase activity.

38. The method of claim 28, wherein the composition further comprises an additional agent that inhibits AKT activity.

39. The method of claim 28, wherein the composition further comprises a farnesyl transferase inhibitor (FTI).

40. A pharmaceutical composition for the treatment of cancer comprising a therapeutically effective amount of a composition comprising a chorionic gonadotropin or a therapeutically active fragment or analogue thereof in combination with a geranylgeranyltransferase inhibitor (GGTI).

41. The composition of claim 40, wherein the chorionic gonadotropin is hCG or a therapeutically active fragment or analogue thereof.

42. The composition of claim 40, wherein the therapeutically active fragment is a beta subunit of hCG.

43. The composition of claim 40, which further comprises an additional agent that inhibits NF-kappa-B activity.

44. The composition of claim 40, which further comprises an additional agent that inhibits PI-3-kinase activity.

45. The composition of claim 40, which further comprises an additional agent that inhibits AKT activity.

46. A method of treating cancer in a subject comprising administering to said subject a therapeutically effective amount of a composition comprising a chorionic gonadotropin or a therapeutically active fragment or analogue thereof in combination with a farnesyltransferase inhibitor (FTI).

47. The method of claim 46, wherein the chorionic gonadotropin is hCG or a therapeutically active fragment or analogue thereof.

48. The method of claim 46, wherein the therapeutically active fragment is a beta subunit of hCG.

49. The method of claim 46, wherein the cancer is selected from the group consisting of, myxoid and round cell carcinoma, Ewing's sarcoma, cancer metastases, including lymphatic metastases, squamous cell carcinoma, esophageal squamous cell carcinoma, oral carcinoma, multiple myeloma, lymphocytic leukemia, acute nonlymphocytic leukemia, chronic lymphocytic leukemia, chronic myelocytic leukemia, and hairy cell leukemia, effusion lymphomas, thymic lymphoma lung cancer, small cell carcinoma of the lungs, cutaneous T cell lymphoma, Hodgkin's lymphoma, non Hodgkin's lymphoma, cancer of the adrenal cortex, non-small cell lung cancers, breast cancer, stomach cancer, colon cancer, colorectal cancer, colorectal neoplasia, pancreatic cancer, liver cancer, bladder cancer, primary superficial bladder tumors, invasive transitional cell carcinoma of the bladder, muscle invasive bladder cancer, prostate cancer, ovarian carcinoma, primary peritoneal epithelial neoplasms, cervical carcinoma, uterine endometrial cancers, vaginal cancer, cancer of the vulva, uterine cancer, solid tumors in the ovarian follicle, testicular cancer, penile cancer, kidney cancer, renal cell carcinoma, brain cancer, neuroblastoma, astrocytic brain tumors, gliomas, metastatic tumor cell invasion in the central nervous system, osteomas, osteosarcomas, malignant melanoma, tumor progression of human skin keratinocytes, basal cell carcinoma, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma.

50. The method of claim 46, wherein the cancer is prostate cancer.

51. The method of claim 46, further comprising the step of treating said cancer with radiation after administration of the chorionic gonadotropin and farnesyltransferase inhibitor composition.

52. The method of claim 51, wherein the radiation is external beam X-ray radiation.

53. The method of claim 51, wherein the radiation is administered by means of brachytherapy.

54. The method of claim 46, wherein the composition further comprises an additional agent that inhibits NF-kappa-B activity.

55. The method of claim 46, wherein the composition further comprises an additional agent that inhibits PI-3-kinase activity.

56. The method of claim 46, wherein the composition further comprises an additional agent that inhibits AKT activity.

57. The method of claim 46, wherein the composition further comprises a granylgeranyltransferase inhibitor (GGTI).

58. A pharmaceutical composition for the treatment of cancer comprising a therapeutically effective amount of a composition comprising a chorionic gonadotropin or a therapeutically active fragment or analogue thereof in combination with a farnesyltransferase inhibitor (FTI).

59. The composition of claim 58, wherein the chorionic gonadotropin is hCG or a therapeutically active fragment or analogue thereof.

60. The composition of claim 58, wherein the therapeutically active fragment is a beta subunit of hCG.

61. The composition of claim 58, which further comprises an additional agent that inhibits NF-kappa-B activity.

62. The composition of claim 58, which further comprises an additional agent that inhibits PI-3-kinase activity.

63. The composition of claim 58, which further comprises an additional agent that inhibits AKT activity.
